SEQUENCE LISTING (76 sequences)

SEQ ID NO: 1 - DNA, 22 bp, Artificial Sequence
  Synthetic oligonucleotide
caaaaggaggcgagtagggtga

SEQ ID NO: 2 - DNA, 23 bp, Artificial Sequence
  Synthetic oligonucleotide
atagtacgtggcacaatgggaga

SEQ ID NO: 3 - DNA, 23 bp, Artificial Sequence
  Synthetic oligonucleotide
taacaagcagcaatgatgagtgc

SEQ ID NO: 4 - DNA, 20 bp, Artificial Sequence
  Synthetic oligonucleotide
aagtgatcttacagccaaca

SEQ ID NO: 5 - DNA, 22 bp, Artificial Sequence
  Synthetic oligonucleotide
gtggctcttatccagaacctag

SEQ ID NO: 6 - DNA, 23 bp, Artificial Sequence
  Synthetic oligonucleotide
ttctttctccacacatccactgc

SEQ ID NO: 7 - DNA, 22 bp, Artificial Sequence
  Synthetic oligonucleotide
caaactgagtgtgacgtaggac

SEQ ID NO: 8 - DNA, 21 bp, Artificial Sequence
  Synthetic oligonucleotide
agtcccagcagcaacagagaa

SEQ ID NO: 9 - DNA, 21 bp, Artificial Sequence
  Synthetic oligonucleotide
tgtgatacaaaggctgccagg

SEQ ID NO: 10 - DNA, 21 bp, Artificial Sequence
  Synthetic oligonucleotide
accagcaggtgtttgggagaa

SEQ ID NO: 11 - DNA, 25 bp, Artificial Sequence
  Synthetic oligonucleotide
tctggagctcaaagaaacatagagt

SEQ ID NO: 12 - DNA, 23 bp, Artificial Sequence
  Synthetic oligonucleotide
cagggctaaatagtaagacagag

SEQ ID NO: 13 - DNA, 23 bp, Artificial Sequence
  Synthetic oligonucleotide
cttggaccttcatattcagtgtg

SEQ ID NO: 14 - DNA, 23 bp, Artificial Sequence
  Synthetic oligonucleotide
tccagagtttcaaatcctccctc

SEQ ID NO: 15 - DNA, 19 bp, Artificial Sequence
  Synthetic oligonucleotide
ctagacaggggatctggct

SEQ ID NO: 16 - DNA, 21 bp, Artificial Sequence
  Synthetic oligonucleotide
cccccttctgaaactgtgtcc

SEQ ID NO: 17 - DNA, 23 bp, Artificial Sequence
  Synthetic oligonucleotide
tagatctgtgcactaagtccgac

SEQ ID NO: 18 - DNA, 21 bp, Artificial Sequence
  Synthetic oligonucleotide
tctgacatctgagagcagtca

SEQ ID NO: 19 - DNA, 24 bp, Artificial Sequence
  Synthetic oligonucleotide
gctagaaatgagtacgtagatcac

SEQ ID NO: 20 - DNA, 21 bp, Artificial Sequence
  Synthetic oligonucleotide
agggccagcctctgctatgtc

SEQ ID NO: 21 - DNA, 22 bp, Artificial Sequence
  Synthetic oligonucleotide
cagttgctgataacctgacagc

SEQ ID NO: 22 - DNA, 22 bp, Artificial Sequence
  Synthetic oligonucleotide
cccagtgttcctgaccaatgtt

SEQ ID NO: 23 - DNA, 21 bp, Artificial Sequence
  Synthetic oligonucleotide
caagcaaggtccaggtcacta

SEQ ID NO: 24 - DNA, 22 bp, Artificial Sequence
  Synthetic oligonucleotide
caatgccacactcacctaacac

SEQ ID NO: 25 - DNA, 23 bp, Artificial Sequence
  Synthetic oligonucleotide
aataagttcttgttcctttcctc

SEQ ID NO: 26 - DNA, 25 bp, Artificial Sequence
  Synthetic oligonucleotide
tttggctgccctaaactctttgtag

SEQ ID NO: 27 - DNA, 21 bp, Artificial Sequence
  Synthetic oligonucleotide
tcactccaccttgagaactct

SEQ ID NO: 28 - DNA, 22 bp, Artificial Sequence
  Synthetic oligonucleotide
gctcagtgaactcagtcctaag

SEQ ID NO: 29 - DNA, 22 bp, Artificial Sequence
  Synthetic oligonucleotide
caaggacactgagagcaacaga

SEQ ID NO: 30 - DNA, 23 bp, Artificial Sequence
  Synthetic oligonucleotide
gaagaggactatgactggctgtt

SEQ ID NO: 31 - DNA, 22 bp, Artificial Sequence
  Synthetic oligonucleotide
cgcgactgaaatgtcctgaatg

SEQ ID NO: 32 - DNA, 25 bp, Artificial Sequence
  Synthetic oligonucleotide
acatgaaggcttacaaccatctgaa

SEQ ID NO: 33 - DNA, 21 bp, Artificial Sequence
  Synthetic oligonucleotide
tacagcggcaaagagaggaag

SEQ ID NO: 34 - DNA, 21 bp, Artificial Sequence
  Synthetic oligonucleotide
aagagagaccccttggtactg

SEQ ID NO: 35 - DNA, 22 bp, Artificial Sequence
  Synthetic oligonucleotide
gaatggatctgcatctgaggtt

SEQ ID NO: 36 - DNA, 23 bp, Artificial Sequence
  Synthetic oligonucleotide
ctcgaacttagacaactgtggtc

SEQ ID NO: 37 - DNA, 21 bp, Artificial Sequence
  Synthetic oligonucleotide
aggcctacagacagttgtgca

SEQ ID NO: 38 - DNA, 21 bp, Artificial Sequence
  Synthetic oligonucleotide
cttccatgctagggctagtga

SEQ ID NO: 39 - DNA, 21 bp, Artificial Sequence
  Synthetic oligonucleotide
cccttaatcaattgctgcaaa

SEQ ID NO: 40 - DNA, 20 bp, Artificial Sequence
  Synthetic oligonucleotide
agccacatgtgcttgctttc

SEQ ID NO: 41 - DNA, 18 bp, Artificial Sequence
  Synthetic oligonucleotide
ctgagtgatgcagggtgc

SEQ ID NO: 42 - DNA, 20 bp, Artificial Sequence
  Synthetic oligonucleotide
ggctgtgaatccagaggctt

SEQ ID NO: 43 - DNA, 20 bp, Artificial Sequence
  Synthetic oligonucleotide
tgtagtcccctggatcccat

SEQ ID NO: 44 - DNA, 22 bp, Artificial Sequence
  Synthetic oligonucleotide
tgccagctcaagttgtcttttg

SEQ ID NO: 45 - DNA, 20 bp, Artificial Sequence
  Synthetic oligonucleotide
gtcagaaggggcagactcta

SEQ ID NO: 46 - DNA, 20 bp, Artificial Sequence
  Synthetic oligonucleotide
tcacttgggatggtgaactg

SEQ ID NO: 47 - DNA, 21 bp, Artificial Sequence
  cagacctcag ggcatttcca t
cagacctcagggcatttccat

SEQ ID NO: 48 - DNA, 21 bp, Artificial Sequence
  Synthetic oligonucleotide
atatggctctttgttctctgg

SEQ ID NO: 49 - DNA, 20 bp, Artificial Sequence
  Synthetic oligonucleotide
cagatctcagggcattccca

SEQ ID NO: 50 - DNA, 21 bp, Artificial Sequence
  Synthetic oligonucleotide
atatggctctttgttctctgg

SEQ ID NO: 51 - DNA, 176 bp, Mus musculus
caaaaggaggcgagtagggtgacattactgtcctaaaaatcaagactctttctttctttctttctttctttctttctttctttctttctttctttctttctttctttctttctttctttctttctttctttctttctatcacagaaaaaatgctctcccattgtgccacgtactat

SEQ ID NO: 52 - DNA, 503 bp, Mus musculus
taacaagcagcaatgatgagtgctttgcaaatatatctgggctattctaaaattgactactataaaccaccccatctatctagacagttttgtttcctggagggcattctgttttgttctgtaatgatatttcaagctgtatgggagtcttgtttccacacgtgactacatttattaacttagccacatctaaaatacagtgtgggaacccagtaagggtatatggtatcttaactctcatccaattttcagctgggagcattgcatgctattttccccttgttttcctttctttctttctttctttctttctttctttctttctttctttctttctttctttctttctttctttctttctaatgtttcttttcaaagattttgaatttgtaatgaacacatgagggtatatatcccaccttgatctagctgtggagatccccttggctactgtggaaaataccactagacaactgtgggagatgttggctgtaagatcactt

SEQ ID NO: 53 - DNA, 484 bp, Mus musculus
gtggctcttatccagaacctagggaggatttaagacatttggaaggtccaaatgcttagctgcaagcattgcacccatttctccctttctttctttctttctttctttctttctttctttctttctttctttctttctttctttctttctttctttctttctttctttctttctttctaattttttattaggtattttcctcatttacatttctaatgctatcccaaaagtcccccatacccacccccccccactcccctacccaccaactctccctttttggccctggcgttcccctgtactggggcatataaagttttgcacccatttcttataaggaacttcagcatttgtggattttggtttgtggtatacaggatcttggaagaatcccttagacaccaaggaactactgactactggacagagtattttgtatgtgtagtagctaggtacccaaggcagtggatgtgtggagaaagaa

SEQ ID NO: 54 - DNA, 180 bp, Mus musculus
caaactgagtgtgacgtaggacaggaataaagatcagaccgcgaggcaaggaggcaaaagaaagaaagaaagaaagaaagaaagaaagaaagaaagaaagaaagaaagaaagaaagaaagaaagaaagaaagaaagaaaagggagagaatctggtggttttctctgttgctgctgggact

SEQ ID NO: 55 - DNA, 317 bp, Mus musculus
tgtgatacaaaggctgccaggcttcagtagagtatatttgccttcctgggtgcctattctgttttcctttctttctttctttctttctttctttctttctttctttctttctttctttctttctttctttctttctttctttctctgagacagggtttctctgtgtagccctggctgtcctggaactcgctttgtagaccaggctggcctcgaactcagtaatccacctgcctctgcctcccaaatgctgggattaaaggcgataacccctttcaacctcattctttgcatgccccttctcccaaacacctgctggt

SEQ ID NO: 56 - DNA, 135 bp, Mus musculus
tctggagctcaaagaaacatagagttattgcaattaatttctttctttctttctttctttctttctttctttctttctttctttctttctttctttcgagagacaggtcttactctgtcttactatttagccctg

SEQ ID NO: 57 - DNA, 144 bp, Mus musculus
cttggaccttcatattcagtgtgggaaagaaagaaagaaagaaagaaagaaagaaagaaagaaagaaagaaagaaagaaagaaagaaagaaagaaagaaagaaagaaagaaaggtaaaaaagagggaggatttgaaactctgga

SEQ ID NO: 58 - DNA, 216 bp, Mus musculus
ctagacaggggatctggctaggaggagtgtttgcagaagaaacaggcattaactcttaactcttgcccaggccactctgtttctagctgggatgacaccatcagaattctttctttctttctttctttctttctttctttctttctttctttctttctttctttctttctttctttctttctttcttcttcttttggacacagtttcagaaggggg

SEQ ID NO: 59 - DNA, 399 bp, Mus musculus
tagatctgtgcactaagtccgacacacaaataaaatacagaacgaaaagcccagaggagagaaagaatcctcaagaaacatccaagaagacattgcagagtgtgaagagtgaagggctctagcccaggcccacatcatggtgtatctagggaaagttttatgagcttctagaaagaaaacaaacacaatcaagacatacatttatttgtttattctttctttctttctctctccttctttctttctttctttctttctttctttctttctttctttctttctttctttctttctttctttctttctttcttcattaattcattcatttatgtgtgtggctgtcttgcctgcatggttatctttataccacatgtgcactgactgctctcagatgtcaga

SEQ ID NO: 60 - DNA, 330 bp, Mus musculus
agggccagcctctgctatgtcgggaattttatgagaccttgtcagaaagaaagaaagaaagaaagaaagaaagaaagaaagaaagaaagaaagaaagaaagaaagaaagaaagaaagaaagaaagggaaagaaagaaaccataggaagtaatttgtgcattaaaatgtgtgttaagtgaatgtagtcatttatccaacccatatttaggtctcatttataaaaacagtgacccagaatatgcatctaacgtgagtagcctgaagccgaagcgcctgtgtacagagtgggttgagagaggatacttggtgatctacgtactcatttctagc

SEQ ID NO: 61 - DNA, 393 bp, Mus musculus
cagttgctgataacctgacagccaaaattccacagagccacataacagaagaagtgaacatacatttacatgcatagtcacttaacacacttaaatagaaaagaaagaaagaaagaaagaaagaaagaaagaaagaaagaaagaaagaaagaaagaaagaaagaaagaaagaaagaaaggaaatagagaaaagagattgccaatatgaaagaagtgactctagcccattcaaaactgtataaaagaatgtctggagcctggctgggggaatggataccaatcatgccaacatcctagaaacctttgctggagccactgacacacccaccaagaaacaagcttcttactgggtgcttctggcctgaggcataaacattggtcaggaacactggg

SEQ ID NO: 62 - DNA, 292 bp, Mus musculus
caatgccacactcacctaacacaggcgtgctgtagaggaactcaactcgaggtccagcctttgcctgaaaacttcccccagacagctgcaagaactcacagtactggtttttgtgtgtctttctttcttttctttctttctttctttctttctttctttctttctttctttctttctttctttctttctttctttccttctttctttttcttatccatgtccaggagctgtcctggacactggctgcttcccaaggagcttcggtagggcatagtgacctggaccttgcttg

SEQ ID NO: 63 - DNA, 136 bp, Mus musculus
tttggctgccctaaactctttgtagtgcccaattgaagaaagaaagaaagaaagaaagaaagaaagaaagaaagaaagaaagaaagaaagaaagaaagaaagaaagaaagaaagaggaaaggaacaagaacttatt

SEQ ID NO: 64 - DNA, 308 bp, Mus musculus
tcactccaccttgagaactctctttgtgcctattagatttttcattttagatctttcttcatttttataccagtgttgtggttgagttttgtttctattgttttttccatgaatttgaccaagaaaagccctgggaaagaatttctttctttctttctttctttctttctttctttctttctttctttctttctttctttctttctttcttttttttttttaagctagctgcatacacttgcatatcttgccctaggctgtgggacaataaacttacttttctaaccttaggactgagttcactgagc

SEQ ID NO: 65 - DNA, 505 bp, Mus musculus
caaggacactgagagcaacagagaggccctctgttgctgaggagtctctatcccaactcagcccccaacactgagcatctccctcacaatttccatcccagacccccataataacaggaggggcctagggagcccttcctactctcttgaataccaccaataaagttcgctgcacagaaagaaagaaggaaagaaagaaagaaagaaagaaagaaagaaagaaagaaagaaagaaagaaagaaagaaagaaagaaagagctaacctctggtatttccccagactgaactctcagagttggaagagactcccaaggtcacattgcccattctcttactttatggataagaaagggtggttcagagaacttgaaatagccctcaggcagtgcgtggcaggaggattttgacctcaacctgatcttttctccataatcagtgggcagtgatctaccaccaccttctgcttgtgaggcagacatgaaacagccagtcatagtcctcttc

SEQ ID NO: 66 - DNA, 245 bp, Mus musculus
acatgaaggcttacaaccatctgaacagctacagtatactcacatacaataaataaataaataaataaataaataaacaaataaataaatcttttaagaaagaaagagagagaggaagaaaggaagaaagaaagaaagaaagaaagaaagaaagaaagaaagaaagaaagaaagaaagaaagaaagaaacaggatttgtatctagaaatgcctacatgagttgcattcaggacatttcagtcgcg

SEQ ID NO: 67 - DNA, 396 bp, Mus musculus
tacagcggcaaagagaggaagaaagaaagaaagaaagaaagaaagaaagaaagaaagaaagaaagaaagaaagaaagaaagaaagacagacaaatgcagatttctaaaatttaggagtaactgaagtatatttttaaaatgcaaaattggtcctgtttttatgggaacactaatggcaactcaaccccagataatttttcttatttattttatctatctatctatctatttatttatttatttatttattactagatattttctttatatacatttcaaatgctatcctgaaagttccctatactctccctctgccctgctcccctaaccacccactcccgcttcttggccctgtactggggcatataaagtttgcagtaccaaggggtctctctt

SEQ ID NO: 68 - DNA, 283 bp, Mus musculus
gaatggatctgcatctgaggttttcttccttcctccctccctccctcccttgcttctttctttctttctttctttctttctttctttctttctttctttctttctttctttctttctttctttctttctttctttctttccttcatgtatcctgtctgtttttaagattagccacatagagagcaatttgataacagattggagtacttctgtgagtctgccctccagctgactaaaaccctgattgctaggaaagaatggaccacagttgtctaagttcgag

SEQ ID NO: 69 - DNA, 365 bp, Mus musculus
aggcctacagacagttgtgcagtatagtgggtgctgagaatcaaagctggtcctctagaagagcagctagcttgttttcttcctttctttctttctttctttctttctttctttctttctttctttctttctttctttcctgtaagtacactgtagctgttttcagacactccagaggagggcgccagatctcattacagatggttgtgagcaacagtgtggttgctgggaattgaactcagaatcttcagaagagcaagttagtgctcttaaccactctgccatctctccagccccccatccccaactagctagttttcttgagaacattatgactagcccaatcactagccctagcatggaag

SEQ ID NO: 70 - DNA, 140 bp, Mus musculus
agccacatgtgcttgctttctttctttctttctttctttctttctttctttctttctttctttctttctttctttctttctttctttctttctttctttatcagaaacaagagtatccatttgcagcaattgattaaggg

SEQ ID NO: 71 - DNA, 272 bp, Mus musculus
ctgagtgatgcagggtgctactctgcatctgtgccagaattgcagaaggttttctttctttctttctttctttctttctttctttctttctttctttctttctttctttctttctttctttctttctttcttttaactgttgggagtgcccacttggcttagtgattcaaaacaggtcctgaactacaggaaatcagctcttccttgaatgagcttgcatagaaggaggaccctgatcctaactgataagaaaagcctctggattcacagcc

SEQ ID NO: 72 - DNA, 347 bp, Mus musculus
tgtagtcccctggatcccatagtctagagtgacacataaatatgaaaatgtttatgcaaaaatgcacagaggagagaaaggaaacaatgagaaatagaaagagagagagatggagaaagatagagacagagagacaaatatagtgaaagagagaaagaaagaaagaaagaaagaaagaaagaaagagagaaagaaagaaagagcacaataggatgcagaagaaatggattgagcaatattgtctcagagtataaggaacaaagaacaggatgaaggataatgacccaagagacaaaagacttatcaattcaccacactgaatggtcaaaagacaacttgagctggca

SEQ ID NO: 73 - DNA, 241 bp, Rattus norvegicus
gtcagaaggggcagactctaaaaccttcaaagctatgcactgtagacttttgaatactgctttctaaggacatcttaacagaaatgcggtttgtttattgaatttattctttccttccttccttctttctttctttctttctttctttctttctttctttctttctttctttctttctttctttctttctttctttttatactccagatttaattcccttccagttcaccatcccaagtga

SEQ ID NO: 74 - DNA, 246 bp, Rattus norvegicus
tcacttgggatggtgaactggaagggaattaaatctggagtataaaaagaaagaaagaaagaaagaaagaaagaaagaaagaaagaaagaaagaaagaaagaaagaaagaaagaaagaaggaaggaaggaaggaaagaataaattcaataaacaaaccgcatttctgttaagatgtccttagaaagcagtattcaaaagtctacagtgcatagctttgaaggttttagagtctgccccttctgact

SEQ ID NO: 75 - DNA, 230 bp, Rattus norvegicus
cagacctcagggcatttccatattctaagttagcaactatacaagttatgaggactagtacaaaatgcccagcatctatgtatctatgtatctatgtatctatgtatctatgtatctatgtatctatgtatcttctatctatctatctatctatctatctatctatctatctatctatctatctatctatctatcaatcatctaagaaaccagagaacaaagagccatat

SEQ ID NO: 76 - DNA, 176 bp, Rattus norvegicus
cagatctcagggcattcccatattctaagtcaacaactcttcatgttatgaagactagtatgaaatccccagcatctatctatctatctatctatctatctatctatctatctatctatctatctatctatctatctatcgatctatctaagaaaccagaaaacaaagagccatat

(19) United States
(12) Patent Application Publication
(10) Pub. No.: US 2023/0183781 A1
(43) Pub. Date: Jun. 15, 2023

(54) METHOD FOR THE GENOTYPING OF MOUSE STRAINS

(71) Applicant: GVG GENETIC MONITORING GMBH, Leipzig (DE)

(72) Inventors: Peter DOBROWOLSKI, Radebeul (DE); Melina FISCHER, Rötha (DE)

(73) Assignee: GVG GENETIC MONITORING GMBH, Leipzig (DE)

(21) Appl. No.: 16/616,466

(22) PCT Filed: May 22, 2018
(86) PCT No.: PCT/EP2018/063343

§ 371 Date: Nov. 24, 2019

(30) Foreign Application Priority Data
May 24, 2017 (DE) 10 2017 005 000.7

(51) Int. Cl. C12Q 1/6827; C12Q 1/6853; C12Q 1/6888

(52) U.S. Cl. CPC C12Q 1/6827; C12Q 1/6853; C12Q 1/6888; C12Q 2600/16; C12Q 2600/156; C12Q 2600/124

(57) ABSTRACT

The invention relates in general to the analysis of genetic markers in individuals of non-human origin, in particular the genotyping of outbred populations of rodents or fish. The invention relates specifically to the simultaneous amplification of at least five different polymorphic autosomal markers and at least one polymorphic Y-chromosomal marker of the mouse in a reaction preparation by means of polymerase chain reaction or other multiplex methods and the detection of the specific alleles for each marker of the multiplex method. The invention further relates to a kit for genetically identifying and/or for differentiating two or more animals from DNA extracts of individuals from wild-type populations, from different inbred or outbred strains, of an identical inbred or outbred strain, or from substrains of an identical inbred or outbred strain, in particular of the rat or mouse.

### FIELD OF THE INVENTION

The present invention relates in general to the analysis of genetic markers in individuals of nonhuman origin, especially to the genotyping of outbred populations of rodents or fishes. The invention specifically relates to the simultaneous amplification of at least 5 different polymorphic autosomal markers and at least two polymorphic Y-chromosomal markers of mouse in a reaction mix with the aid of the polymerase chain reaction or other multiplex methods and the detection of the specific alleles for each marker of the multiplex method. The invention further relates to a kit for the genetic identification and/or for the distinguishing of two or more animals from DNA extracts of individuals from wild-type populations, from different inbred or outbred strains, of the same inbred or outbred strain or of substrains of the same inbred or outbred strain, especially of a rat or mouse.

### PRIOR ART

Animal experiments are still an indispensable part of modern research. With the goal of ensuring reproducible experiments, what is indispensable is not only the standardization of breeding conditions in animal husbandry, but also the standardization of the genetic quality of the test animals used. For the breeder of such animals, this means the use of defined systems for the stabilization of genetic structures. Such structures are intended to ensure a standardization over the period of many generations and over geographical distance. The latter also concerns the provision of test strains with identical designation from different breeding sites and/or from different breeders.

Inbred strains are mouse and rat models in which the genetic diversity among the individuals has been lost and is thus negligible owing to consistent brother—sister mating over many generations. Animals of an inbred strain or substrain of an inbred strain are genetically identical to an extent of over 99.9% and moreover homozygous. Experiments with such animals allow the statistical validity of experimental data, since they can be collected simultaneously on a multiplicity of effectively identical twins. However, such animal models are of only limited informative value for the application of experimental findings to humans, since what are represented here are artificial (inbred) populations which do not occur in this way in nature.

In parallel to inbred strains, there is also the necessity of the availability of standardized strains in which the genetic diversity has been maintained. Examples of such strains are mosaic and outbred populations. They serve as a practical model for noninbred populations, as typically also occur in animals and humans. With the aid of outbred populations, the aim is to represent the genetic variation within a population and to utilize said variation in relevant experiments. The genotype of each outbred animal within an outbred population is unique and is not repeated in a second animal. The goal of outbred populations is to maintain a defined, highest possible genetic heterogeneity while observing certain limits of variation within a closed, genetically well-characterized population over the course of generations. Such outbred strains exist for mouse, rat and zebrafish.

A high genetic variability can, for example, be achieved by crossing various, little-related strains or inbred strains during the setup phase of an outbred population. Thereafter, the population is closed, and only matings within the population are then allowed. In contrast to inbred strains, parent animals related to one another are not mated with one another. This is to ensure a stable and standardized genetic variability. The major requirements for an outbreeding system have been summarized by Rapp (1972): (1) maximum maintenance of population-specific allele and genotype frequencies, (2) minimization of increase in homozygosity and degree of inbreeding, (3) avoidance of formation of sublines, (4) ease of use. Strictly speaking, what is concerned is a genetically well-characterized, but limited population of animals that is composed of individuals which originally originate from different strains. Therefore, the technically correct designation is “outbred population”. Proceeding from the designation “inbred strains” and to illustrate the diametrical difference between the principles of outbreeding and inbreeding, the literature frequently also utilizes the designation “outbred strain”, and this is therefore a synonym for “outbred populations”.

For the reliable reproducibility of test results with outbred animals, a representative cross section of the genotype distribution of the specific outbred population would have to be available for each experiment. If no genetic markers are used to describe the genetic diversity, this represents a huge challenge for the breeder. For the user, the situation is even more complicated. Frequently, only very limited animal numbers are ordered from the breeder for the individual test stages and it is not possible at all to check whether the individual animals originate from a single (e.g., a parent pair) breeding group or different breeding groups of the outbred population.

The original creation and structuring of outbred populations involves crossing different male founder animals which, for their part, each introduce their individual variant of the Y chromosome into the outbred population. If the outbreeding is conducted correctly, all the originally available variants remain in the population. In different outbred populations and also within a defined outbred population, there is likely to be a multiplicity of different Y chromosomes which may also exhibit considerable genetic differences. Although a major aspect for defining the genetic diversity of an outbred population, no scientific data at all are available to date for the number of different Y chromosomes.

In the case of the inbred strain currently utilized in research, the Y chromosome goes back to one of the two types Mus musculus musculus (M. m. musculus) and Mus musculus domesticus (M. m. domesticus), which separated from one another approx. 900 000 years ago. Genetic analyses have revealed that the mouse species M. m. musculus, M. m. castaneus and M. m. molossinus all bear a Y chromosome of the type M. m. musculus. By contrast, M. m. domesticus represents a separate, second basic type (Pertile et al., 2009).

It is known that the Y chromosome in the case of mouse acts as a global regulator of genome-wide expression and that the crossing of a strain-exogenous Y chromosome often leads to considerable phenotypic changes in male and female descendants (Nelson et al., 2010). Therefore, different variants of the Y-chromosome represent a crucial parameter of the genetic and phenotypic variation of an outbred population. Describing genetic diversity thus also requires knowledge of the number and the nature of different variants of the Y chromosome in the population. For outbred strains in the case of mouse, neither the number of different Y-chromosomal haplotypes nor their belonging to M. m. musculus or M. m. domesticus is known to date.

Whereas a large marker repertoire of DNA polymorphisms is utilized for inbreeding in the case of mouse, there are to date no standardized monitoring programs or marker sets for outbreeding of mouse (Kluge and Wedekind, 2016). Yalcin et al. (2010) describe the parallel utilization of multiple 100 000 SNPs for the genetic characterization of outbred populations of mouse.

Microsatellites, also called short tandem repeats (STRs), are used for the genotyping of inbred strains. These are short DNA sequences of 1 to 6 bases in length, the basic motif of which is repeated multiple times like beads strung together, for example [CA]n, [GAC]n or [GATA]n. Owing to the different number of such repeats in different individuals, it is possible to distinguish such individuals from one another. Witmer et al. (2003) describe the utilization of STRs having a dinucleotide repeat unit to distinguish the various inbred strains of mouse. US 2014/0066322 describes the use of a PCR multiplex to distinguish cell lines in the case of mouse, in which 9 different mouse STRs are combined with 2 further human STR markers. WO 2016/008894 describes genotyping to distinguish individuals within inbred strains. Both applications use STR markers, the repeat unit of which consists of 4 nucleotides.

All the available STR data collected for mouse are based on the analysis of inbred strains. The utilization of STRs for outbred populations of mouse is unknown. Biostatistical parameters for assessing the quality of individual mouse STR markers, such as polymorphism information content (PIC) or heterozygosity, are unavailable. Shang et al. (2014) describe 6 STRs which were utilized for genotyping in the case of rat for the outbred strains Wistar and Sprague Dawley.

The genetic monitoring of outbred populations in the case of mouse, rat or zebrafish is therefore an area which can still be distinctly optimized (Kluge and Wedekind, 2016). This requires meaningful biostatistical parameters, which are unavailable to date. It would be advantageous if a method based on autosomal and sex-specific STR markers were to be available, allowing the genotyping and comprehensive biostatistical characterization of outbred populations. A suitable method is the multiplex STR analysis of polymorphic tetranucleotide STR loci of mouse, and this is the subject matter of the present invention.

### SUMMARY OF THE INVENTION

It is therefore an object of the invention to overcome the disadvantages of the prior art and to make reliable means and methods for genotyping outbred populations available to the breeder and user.

This object is achieved by providing particularly suitable STR markers, with the aid of which a set of markers for genotyping outbred populations can be made available to the breeder and user.

It is further an object of the invention to enable the breeder to genetically characterize outbred populations, to document the genetic diversity and to carry out a genetic monitoring of important biostatistical parameters during breeding. It is further an object of the invention to make available a method for assessing the genetic diversity within an outbred population on the basis of autosomal and Y-chromosomal markers. It is likewise an object of the invention to allow a comparative assessment of the quality of outbred populations with one another through the use of a uniform set of markers. Furthermore, it is intended to allow the user to be able to independently assess the genetic diversity of the outbred animals purchased from commercial breeders.

To achieve the abovementioned objects, the invention provides, in one aspect, a method for genetically identifying and/or for distinguishing two or more animals from different wild-type populations, from different outbred strains, of the same outbred strain or of substrains of the same outbred strain of the species mouse, rat, hamster and zebrafish, the method comprising the simultaneous amplification of at least 5, preferably at least 6, 7 or 8, particularly preferably at least 9 or 10, most preferably at least 11 different polymorphic autosomal markers and at least two polymorphic Y-chromosomal markers of the respective species in a reaction mix with the aid of the polymerase chain reaction or other multiplex methods and, optionally, the detection of the specific allele for each marker of the multiplex method.

The method likewise comprises providing relevant oligonucleotide primer pairs, with each marker being assigned a primer pair which specifically hybridizes on both sides of the marker region. The method further describes the simultaneous amplification of at least 7, preferably at least 8, 9 or 10, particularly preferably at least 11 or 12, most preferably at least 13 of the selected markers in a multiplex reaction mix and the formation of a mixture of alleles with reaction products for each of the participating markers.

The method further comprises the detection of the individual alleles in the allele mixture and the unambiguous assignment thereof to a marker and to a defined allele for the specific marker.

In a separate embodiment of the present invention, a method allowing the assignment of the Y chromosome of outbred populations to the type M. m. musculus or M. m. domesticus with the aid of STR markers is provided.

In a further embodiment of the present invention, a method allowing the identification of different variants of the Y chromosome within an outbred population of mouse with the aid of STR markers is provided.

In a separate embodiment of the present invention, a method allowing the identification of different variants of the Y chromosome of rat with the aid of STR markers is provided.

The marker set according to the invention can additionally be supplemented by those markers which allow an assignment to individual mouse strains. Suitable for this purpose are, for example, known strain-specific insertion-deletion polymorphisms (indels) or SNPs. As an example of such markers, the 25 bp deletion in the gene Disc1 for the strain 129, as described by Clapcote and Roder (2006), can be mentioned. These additional markers are particularly suitable for the use of the inventive STR multiplex assays in assignment of founder animals of an outbred population to known inbred strains, the characterization of transgenic lines of mouse and also the testing of the origin of cell lines.

With the present invention, what is made available in a further aspect is a kit which can be used to detect the alleles of a marker set that are present in a DNA sample. The kit contains all the necessary components for carrying out a simultaneous coamplification and for detecting alleles with the involvement of at least 5, preferably at least 6, 7 or 8, particularly preferably at least 9 or 10, most preferably at least 11 different autosomal STR markers and at least two, preferably three Y-chromosomal STR markers for at least one DNA sample.

The following detailed description of the invention and the attached FIGURE and exemplary embodiments are intended to more particularly elucidate the essence of the invention, to show further possible uses and to demonstrate the the advantages arising therefrom.

In a further aspect, the invention provides a kit for the genetic identification and/or for the distinguishing of two or more animals from different strains, of the same strain or of substrains of the same strain of the of the species mouse, rat, hamster and zebrafish from DNA extracts, the kit comprising:
(a) a set of oligonucleotide primer pairs in which each primer pair binds specifically to the flanking DNA segments of a locus from the set of STR loci and which comprises at least 5, preferably at least 6, 7 or 8, particularly preferably at least 9 or 10, most preferably at least 11 autosomal STR markers which can be coamplified simultaneously in a reaction mix;
(b) a set of oligonucleotide primer pairs which bind specifically to the flanking DNA segments of the Y chromosome and which comprises at least two, preferably at least three Y-chromosomal STR markers which can be coamplified simultaneously with the autosomal STR loci (a) in a reaction mix;
(c) reagents which are sufficient for carrying out at least one multiplex PCR; and
(d) a size standard which contains different DNA markers of a known fragment length.

In a preferred embodiment of the invention, the kit consists of parts (a) to (d).

### DETAILED DESCRIPTION OF THE INVENTION

### Definitions

Allele: Allele refers to various forms of a gene or a gene sequence in a defined DNA region of a chromosome.

Amplification: Amplification describes the reproduction of DNA segments. This can occur naturally or else be generated artificially (in vitro). The latter is, for example, realized in molecular biology via the PCR method.

Haplotype: Haplotype describes a haploid genotype, a unique variant of a nucleotide sequence on the same chromosome. The Y chromosome occurs in the genome only singly and cannot exchange DNA segments with another chromosome, meaning that the Y-chromosomal haplotype remains constant.

Multiplex: In contrast to a singleplex PCR, in which only one primer pair is utilized, a multiplex PCR mix contains a plurality of different primer pairs, with each of the primer pairs amplifying a different region of the chromosomal DNA.

PCR: The polymerase chain reaction is a method which is used to reproduce DNA in vitro with the aid of DNA polymerases (e.g., Taq polymerase). The starting point used for the new synthesis are specific primers which bind specifically to defined DNA segments.

Polymorphic: A marker is polymorphic if it has at least two or more different alleles.

Primer/oligonucleotide primer: In molecular biology, primer refers to an oligonucleotide which serves as the starting point for the amplification of DNA segments.

### Preferred Embodiments of the Invention

Particularly good results were achieved in the genotyping of outbred populations when the set of loci for the DNA sample, from which the autosomal STR markers are selected, comprises at least 11 autosomal STR markers which can be coamplified simultaneously in a reaction mix.

According to a first aspect, the invention therefore provides a method for genetically identifying and/or for distinguishing two or more animals from different wild-type populations, from different outbred strains, of the same outbred strain or of substrains of the same outbred strain of the species mouse, rat, hamster or zebrafish, comprising the following substeps
(a) using at least one DNA sample which is to be analyzed,
(b) selecting a set of loci for the DNA sample which comprises at least 11 autosomal STR markers which can be coamplified simultaneously in a reaction mix,
(c) selecting a set of loci for the DNA sample which comprises at least two Y-chromosomal STR markers which can be coamplified simultaneously with the autosomal STR loci (b) in a reaction mix,
(d) using a set of oligonucleotide primer pairs in which each primer pair binds specifically to the flanking DNA segments of an STR marker from the set of the STR loci,
(e) coamplifying the loci selected for the set, the reaction product being a mixture consisting of amplified alleles of each of the coamplified STR loci of the set, and
(f) analyzing the allele mixture with the goal of determining and assigning the specific alleles for each of the amplified loci of the set for the given DNA sample;
   the autosomal STR loci being characterized by a high value for the “polymorphism information content”, which is within the range of 0.30 and 0.99, preferably within the range of 0.50 to 0.99, preferably within the range between 0.60 and 0.99.

In a preferred embodiment of the invention, the method for genetically identifying and/or for distinguishing two or more animals from different wild-type populations, from different outbred strains, of the same outbred strain or of substrains of the same outbred strain of the species mouse, rat, hamster and zebrafish consists of substeps (a) to (f).

In a further preferred embodiment of the method according to the invention, the DNA sample originates from at least one animal of the species mouse, rat, hamster or zebrafish or from a cell line of the species mouse, rat, hamster or zebrafish.

One advantage of the invention is that the use of the method according to the invention is in no way limited only to outbred strains. It can be utilized in all cases in which a comparative DNA analysis of genetic markers is meaningful, such as, for example, for the authentication of cell lines, and the genetic characterization of captured wild animals (wild-type populations), of inbred strains and of genetically modified lines of mouse. For example, it is possible to utilize a multiplex, containing in each case one marker per chromosome, for monitoring the ploidy of cell lines and for assessing the need to carry out a karyotyping of a cell line.

The present invention concerns the requirement of monitoring the genetic quality of outbred populations, as already elucidated at the start. This requires meaningful biostatistical parameters, which can be collected both by the breeder and by the user as a result of a genotyping procedure. A particular advantage of the invention is that the inventive multiplex assay based on STR markers having tetranucleotide repeat units can be utilized for sufficient genetic characterization and comparison of any desired outbred strains of mouse, rat, hamster or zebrafish, preferably of mouse or rat, particularly preferably of mouse. As a result, the genetic monitoring of outbred populations in the case of mouse, rat, hamster or zebrafish, preferably mouse or rat, particularly preferably mouse, is considerably simplified.

In one embodiment, the present invention provides a method which allows the assignment of alleles of defined STR markers in a DNA sample. This method comprises, in a preferred embodiment, the selection of a DNA sample intended for the analysis and the selection of at least 11 autosomal STR markers from a group of polymorphic STR markers of mouse: D1Mmu121, D2Mmu008, D3Mmu158, D4Mmu155, D5Mmu108, D6Mmu120, D7Mmu003, D8Mmu127, D9Mmu100, D10Mmu043, D11Mmu030, D12Mmu056, D13Mmu096, D14Mmu074, D15Mmu084, D16Mmu030, D17Mmu041, D18Mmu069, D19Mmu008. The individual autosomal markers are, according to the invention, located on different chromosomes, and so they are not genetically coupled to one another. This has the advantage that the use of simple biostatistical parameters for describing a population is made possible as a result.

In a preferred embodiment of the method according to the invention, in step (b), at least two of the eleven autosomal STR loci are selected from a group of loci, the group comprising the loci D1Mmu121, D2Mmu008, D3Mmu158, D4Mmu155, D5Mmu108, D6Mmu120, D7Mmu003, D8Mmu127, D9Mmu100, D10Mmu043, D11Mmu030, D12Mmu056, D13Mmu096, D14Mmu074, D15Mmu084, D16Mmu030, D17Mmu041, D18Mmu069 and D19Mmu008.

In a preferred embodiment of the method according to the invention, in step (b), the set of eleven autosomal loci consists of the loci D2Mmu008, D3Mmu158, D4Mmu155, D6Mmu120, D7Mmu003, D8Mmu127, D10Mmu043, D13Mmu096, D14Mmu074, D16Mmu030 and D18Mmu069.

Furthermore, the method according to the invention comprises selecting at least two Y chromosome-located STR markers from a group of possible mouse STR markers: DYMmu001, DYMmu002 and DYMmu003. The marker DYM003 is utilized in order to allow an assignment to the type M. m. musculus or M. m. domesticus. At least one further polymorphic STR marker from DYMmu001 or DYMmu002 is used for distinguishing different haplotypes.

Preferably, the set of loci in step (c) of the method according to the invention is situated on the Y chromosome and can advantageously be utilized for sex identification in the DNA sample of the individual in step (a). A further advantage of the set of loci according to step (c) is that said set of loci for sex identification can distinguish between the Y chromosomes of the type Mus musculus musculus and of the type Mus musculus domesticus.

In a further preferred embodiment, the locus for sex determination is a polymorphic STR locus of mouse and is selected from the group comprising the loci DYMmu001, DYMmu002 and DYMmu003. Such a locus has the advantage that it can distinguish Y-chromosomal haplotypes of mouse.

In a further preferred embodiment, the locus for sex determination is a polymorphic STR locus of rat and is selected from the group comprising the loci DYRno004, DYRno165 and DYRno304. Such a locus has the advantage that it can distinguish Y-chromosomal haplotypes of rat.

This means that it is possible to perform, within a specific outbred population, an assignment of male individuals to different haplotypes and also the assignment to one of the two basic Y chromosome types M. m. musculus or M. m. domesticus. This approach for describing the genetic diversity of outbred populations via the Y chromosome is made available for the first time by the present invention and can be utilized in future for genetic monitoring.

In a particularly preferred embodiment, in step (e) of the method according to the invention, a multiplex amplification with at least thirteen pairs of oligonucleotide primers, which comprises at least eleven autosomal and two Y-chromosomal STR loci, is carried out.

The amplified alleles are preferably separated by means of an analytical or semipreparative method before the evaluation in step (f). A preferred separation method is gel electrophoresis. Particular preference is given to polyacrylamide gel electrophoresis or capillary gel electrophoresis.

Furthermore, it has been found to be advantageous when the primer pairs are labeled for the subsequent detection method. In a preferred embodiment of the method according to the invention, at least one oligonucleotide primer of a primer pair is therefore covalently coupled to a detection dye, preferably to a fluorescent dye.

It is particularly preferred when at least four different dye-coupled primers with four different fluorescent dyes are used. This simplifies the analysis of the allele mixture in step (f), in which the specific alleles are determined and assigned for each of the amplified loci of the set for the given DNA sample. A direct detection of the alleles is thus possible.

The fluorescent dyes can, for example, be selected from the groups of phycobilins, rhodamine and safranins. Suitable fluorescent dyes can, for example, be selected from the group consisting of acridine yellow, acridine orange, aequorin, aesculin, allophycocyanin, 7-aminoactinomycin, ATTO dyes, auramine O, berberine, 9,10-bis(phenylethynyl)anthracene, calcein, 6-carboxyfluorescein, quinine, coumarin dyes, cyanines, 4′,6-diamidino-2-phenylindole, 2,7-dichlorofluorescein, 9,10-diphenylanthracene, eosin B, eosin Y, epicocconone, ethidium bromide, 6-FAM-phosphoramidite, flavins, fluorene, fluorescein, fluorescein arsenic helix binders, fura-2, furaptra, green fluorescent protein (GFP), Hoechst 33342, IAEDANS, Indian yellow, indocyanine green, luciferins, merbromin, N-methylacridone, Nile blue, Nile red, phycocyanin, phycoerythrin, propidium iodide, pyranine, rhodamine B, rubrene, safranin T, stilbene, SYBR green I, SYPRO orange, SYPRO red, SYPRO ruby, Texas red, TMRM+, umbelliferone and xylenol orange.

The fluorescent dyes are preferably covalently bonded to the nucleic acids in the sample.

Lastly, it is advantageous when the assignment of the amplified alleles in step (f) is done on the basis of comparison with a size standard. In a further preferred embodiment of the method according to the invention, what is therefore done is the assignment of the amplified alleles in step (f) on the basis of comparison with a size standard, the size standard being a mixture of DNA fragments of known size and/or a locus-specific mixture of known alleles.

In a further aspect, the invention provides a kit for the genetic identification and/or for the distinguishing of two or more animals from different strains, of the same strain or of substrains of the same strain from DNA extracts, comprising:
(a) a set of oligonucleotide primer pairs in which each primer pair binds specifically to the flanking DNA segments of a locus from the set of STR loci and which comprises at least 11 autosomal STR markers which can be coamplified simultaneously in a reaction mix, consisting of the loci: D2Mmu008, D3Mmu158, D4Mmu155, D6Mmu120, D7Mmu003, D8Mmu127, D10Mmu043, D13Mmu096, D14Mmu074, D16Mmu030, D18Mmu069
(b) a set of oligonucleotide primer pairs which bind specifically to the flanking DNA segments of the Y chromosome and which comprises at least three Y-chromosomal STR markers which can be coamplified simultaneously with the autosomal STR loci (a) in a reaction mix, consisting of the loci DYMmu001, DYMmu002 and DYMmu003
(c) reagents which are sufficient for carrying out at least one multiplex PCR
(d) a size standard which contains different DNA markers of a known fragment length; the animals preferably being strains of mouse.

In a particularly preferred embodiment, the kit further comprises
(e) a size standard which contains a locus-specific mixture of known alleles.

In a further preferred embodiment of the invention, the kit for the genetic identification and/or for the distinguishing of two or more animals from different strains, of the same strain or of substrains of the same strain of mouse from DNA extracts consists of parts a. to d.

The present invention thus provides a method and a kit for genotyping individuals in the case of animal species which are bred in the form of outbred populations, specifically of outbred populations in the case of mouse or rat. It allows the genotyping of individuals, the identification of different haplotypes of the Y chromosome and the description of the genetic diversity of a specific outbred population. The method disclosed here and the kit allow the simultaneous analysis of genomic DNA segments in the case of preferably mouse with the aid of STR markers based on tetranucleotide repeat units, with coamplification of at least eleven different autosomal STR markers and at least two further STR markers for the Y chromosome in a reaction mix.

### General Description of Methods

Regions containing potential STR markers were identified with the aid of the software FastPCR (PrimerDigital Ltd, Kalendar et al. 2014) by importing into the software the GeneBank-deposited Fasta files of the chromosomes of mouse (version GRCm38.p4) and rat (version Rnor_6.0). Regions containing potential STR markers were identified using the module “SSR Search”. The selection criterion was the presence of a tetranucleotide repeat unit which had to be present repeatedly in tandem at least 10 times. By means of PrimerBLAST, primers suitable for the analysis of the markers in the singular approach were generated.

Genomic DNA was extracted from tail-tip biopsies or ear punches with the aid of the NucleoSpin Tissue Kit (Macherey-Nagel, Duren, Germany) according to the information specified by the manufacturer.

On the basis of the analysis of 150 individuals from various inbred strains and outbred populations and of substrains of inbred strains of mouse, the alleles determinable in the DNA samples were determined for 240 different STR markers. Not all candidate markers led to amplifiable DNA products in all the tested strains. Therefore, a further criterion for the selection of suitable markers was that they can be used in all outbred populations and in all inbred strains.

The suitability of the STR markers for describing the genetic diversity of outbred populations was tested on the basis of the genetic analysis of altogether 70 different male individuals of outbred strains CD1, SWISS and NMRI via the determination of the biostatistical parameters polymorphism information content (PIC) and heterozygote rate (Het). For each chromosome, what was selected was that STR marker which has the highest PIC value.

For all STR markers, oligonucleotide primers suitable for the simultaneous amplification of at least 11 different autosomal STR markers were identified. Surprisingly, many of the PrimerBLAST-generated primers were not optimally suitable for multiplex applications. The subsequent optimization of the oligonucleotide sequences with respect to multiplexing ability was done manually without software by testing a multiplicity of different primers. All primers are used at an annealing temperature of 58° C. Forward primers were coupled to one of the fluorescent dyes 6FAM™ (blue), DY530™ (green), DY510XL™ (red), ATTO550™ (yellow) (Eurogentec, the Netherlands). Primers were synthesized by biomers (biomers.net GmbH, Ulm, Germany) and Eurogentec (Eurogentec GmbH, Cologne, Germany).

Table 1 contains the list of the inventive autosomal and Y-chromosomal STR markers of mouse, and the oligonucleotide primers used for the amplification in the multiplex method. For each primer, what is specified is the chromosomal location thereof in base pairs, which was ascertained on the basis of reference DNA (NCBI, version GRCm38.p4). Designating the STR markers has the aim of including essential characterizing data simply on the basis of the name: “D” stands for DNA; the naming of the chromosome (indication directly after the “D”, 1 to 19 for the autosomes and X and Y for the sex chromosomes); identification of the origin of the DNA (e.g., Mmu—Mus musculus, Rno—Rattus norwegicus, Dre—Danius rerio); and the approximate position on the chromosome (in megabases). The STR marker D1Mmu121 is, for example, a DNA marker of of mouse that is situated on chromosome 1 in the region of approx. 121 megabases.

Furthermore, Table 1 shows the structure of the repeat unit for each marker. The indicated number of repeat units is based on the reference sequence for the strain C57BL/6J as deposited in the NCBI database. The alleles were named according to the guidelines for the standardization of nomenclature for STR markers in forensics (DNA Recommendations, 1997).

The primers belonging to a marker are mixed together in the ratio of 1:1 at a concentration of 5 pmol/μl. The PCR was carried out in a total volume of 25 μl using Snooplex® FastPrep PCR reagents (GVG Genetic Monitoring, Leipzig). The reaction mix for the PCR analysis of STR markers in the singular approach consisted of the following components: 16.2 μl of nuclease-free water, 5 μl of 5×PCR buffer, 1.0 μl of primer mixture, 0.8 μl of Taq DNA polymerase and 2 μl of DNA. In the case of the multiplex PCR approach according to the invention, primer mixtures of the following amounts were used: D2Mmu008 (0.8 μl), D3Mmu158 (1.2 μl), D4Mmu155 (0.8 μl), D6Mmu120 (1.6 μl), D7Mmu003 (1.2 μl), D8Mmu127 (1.2 μl), D10Mmu043 (0.4 μl), D13Mmu096 (0.7 μl), D14Mmu074 (1.6 μl), D16Mmu030 (1.6 μl), D18Mmu069 (1.2 μl), DYMmu001 (1.0 μl), DYMmu002 (0.8 μl), DYMmu003 (1.0 μl). Top-up to 25 μl final volume was done by addition of 2.1 μl of nuclease-free water, 5 μl of 5×PCR buffer, 0.8 μl of Taq DNA polymerase and 2 μl of DNA.

The PCR amplification was carried out using a Bio-Rad C1000 Touch thermal cycler (Bio-Rad Laboratories, Hercules, USA). The PCR parameters were 94° C. for 2 min, followed by 32 cycles at 94° C. for 30 s, 58° C. for 1 min, 68° C. for 2 min and a final elongation step at 68° C. for 10 min. The amplified PCR products were analyzed on an ABI 3500 Genetic Analyzer (Applied Biosystems, Foster City, USA). To this end, 1.8 μl of the PCR mixture and 0.2 μl of an internal size standard (MapMarker Custom, BioVentures Inc., Murfreesboro, USA; fragment sizes: 60, 80, 90, 100, 120, 140, 160, 180, 200, 220, 240, 250, 260, 280, 300, 320, 340, 360, 380, 400, 425, 450, 475, 500, 525 and 550 bp labeled with the fluorescent dye Dy-632) were added to 12 μl of deionized formamide. The mixture was injected (15 s) and subsequently resolved at 15 kV and a running temperature of 60° C. in Performance Optimized Polymer 4 (POP4, Applied Biosystems). The analysis data were evaluated using GeneMapper v5 (Applied Biosystems).

The creation and structuring of outbred populations involves using only a limited number of founder animals. Therefore, not all STR-marker alleles which occur in nature in wild mice will also find their way into an outbred colony. Outbred populations thus have, in comparison with wild mice, a limited, defined number of different alleles. This number can vary between the various outbred populations, depending on which founder animals with which alleles were originally used. It is an object of the present invention to identify STR markers which exhibit, in different outbred populations, as many different alleles as possible with relatively uniform frequency distribution. Serving as reference value with respect to the possible number of alleles are genotyping results of over 150 different animals from altogether 17 different inbred strains of mouse.

Assessing an autosomal or X-chromosomal marker requires suitable criteria which describe the quality thereof with respect to genetic diversity and allow a comparison between various markers. Biostatistical parameters suitable for this purpose are “polymorphism information content” (PIC) and heterozygosity (H). Heterozygosity is calculated from the number of heterozygous DNA profiles (two different alleles detectable in the DNA sample) divided by the total number of analyzed DNA profiles. In the case of the calculation of the PIC value, the number of different alleles and also the percentage thereof in the total population is taken into consideration. Markers having a PIC value of 0.50 or more are particularly suitable for describing genetic diversity.

The methods described here and in the exemplary embodiments are merely to be understood as an example. A person skilled in the art is aware of various further possibilities, with the aid of which the goal of the invention can be achieved, such as DNA extraction from sample material, finding STR regions or generating and optimizing specific oligonucleotide primers.

Table 1: List of the inventive autosomal and Y-chromosomal STR markers in the case of mouse, the oligonucleotide primers used, the chromosomal location thereof in base pairs, and the structure of the repeat unit of the STR marker. The number of repeat units is based on the reference sequence for the strain C57BL/6J as deposited in the NCBI database. The alleles were named according to the guidelines (DNA Recommendations, 1997). The genomic DNA sequences of the inventive STR markers are listed in Table 2. (F— forward primer, R— reverse primer)

| | | | Y-chromosomal | |
|---|---|---|---|---|
| | | | position | Ref. sequence, |
| Marker | | Primer sequence (5′-3′) | (bp) | repeat unit |
| D1Mmu121 | | | | |
| SEQ ID NO: | F | CAAAAGGAGGCGAGTAG | 121856210- | (CTTT)22 |
| 1 | | GGTGA | 121856231 | (see SEQ ID NO: 51) |
| SEQ ID NO: | R | ATAGTACGTGGCACAATG | 121856385- | |
| 2 | | GGAGA | 121856363 | |
| D2Mmu008 | | | | |
| SEQ ID NO: | F | TAACAAGCAGCAATGATG | 8370135- | (CTTT)18 |
| 3 | | AGTGC | 8370157 | (see SEQ ID NO: 52) |
| SEQ ID NO: | R | AAGTGATCTTACAGCCAA | 8370637- | |
| 4 | | CA | 8370618 | |
| D3Mmu158 | | | | |
| SEQ ID NO: | F | GTGGCTCTTATCCAGAAC | 158205109- | (CTTT)22 |
| 5 | | CTAG | 158205130 | (see SEQ ID NO: 53) |
| SEQ ID NO: | R | TTCTTTCTCCACACATCC | 158205592- | |
| 6 | | ACTGC | 158205570 | |
| D4Mmu155 | | | | |
| SEQ ID NO: | F | CAAACTGAGTGTGACGTA | 155563079- | (GAAA)20 |
| 7 | | GGAC | 155563100 | (see SEQ ID NO: 54) |
| SEQ ID NO: | R | AGTCCCAGCAGCAACAG | 155563258- | |
| 8 | | AGAA | 155563238 | |
| D5Mmu108 | | | | |
| SEQ ID NO: | F | TGTGATACAAAGGCTGC | 108460247- | (CTTT)19 |
| 9 | | CAGG | 108460267 | (see SEQ ID NO: 55) |
| SEQ ID NO: | R | ACCAGCAGGTGTTTGGG | 108460563- | |
| 10 | | AGAA | 108460543 | |
| D6Mmu120 | | | | |
| SEQ ID NO: | F | TCTGGAGCTCAAAGAAAC | 120379001- | (TTTC)15 |
| 11 | | ATAGAGT | 120379025 | (see SEQ ID NO: 56) |
| SEQ ID NO: | R | CAGGGCTAAATAGTAAGA | 120379135- | |
| 12 | | CAGAG | 120379113 | |
| D7Mmu003 | | | | |
| SEQ ID NO: | F | CTTGGACCTTCATATTCA | 3217283- | (GAAA)22 |
| 13 | | GTGTG | 3217305 | (see SEQ ID NO: 57) |
| SEQ ID NO: | R | TCCAGAGTTTCAAATCCT | 3217426- | |
| 14 | | CCCTC | 3217404 | |
| D8Mmu127 | | | | |
| SEQ ID NO: | F | CTAGACAGGGGATCTGG | 127974039- | (TTCT)20 TCTTCT |
| 15 | | CT | 127974057 | (see SEQ ID NO: 58) |
| SEQ ID NO: | R | CCCCCTTCTGAAACTGTG | 127974254- | |
| 16 | | TCC | 127974234 | |
| D9Mmu100 | | | | |
| SEQ ID NO: | F | TAGATCTGTGCACTAAGT | 100584836- | (TTTC)3N(TCTT)19 |
| 17 | | CCGAC | 100584858 | (see SEQ ID NO: 59) |
| SEQ ID NO: | R | TCTGACATCTGAGAGCA | 100585234- | |
| 18 | | GTCA | 100585214 | |
| D10Mmu043 | | | | |
| SEQ ID NO: | F | GCTAGAAATGAGTACGTA | 43387148- | (GAAA)20GG(GAAA)3 |
| 19 | | GATCAC | 43387125 | (see SEQ ID NO: 60) |
| SEQ ID NO: | R | AGGGCCAGCCTCTGCTA | 43386819- | |
| 20 | | TGTC | 43386839 | |
| D11Mmu030 | | | | |
| SEQ ID NO: | F | CAGTTGCTGATAACCTGA | 30696089- | (GAAA)19 |
| 21 | | CAGC | 30696110 | (see SEQ ID NO: 61) |
| SEQ ID NO: | R | CCCAGTGTTCCTGACCAA | 30696481- | |
| 22 | | TGTT | 30696460 | |
| D12Mmu056 | | | | |
| SEQ ID NO: | F | CAAGCAAGGTCCAGGTC | 56692378- | (CTTT)19CCTT(CTTT)2 |
| 23 | | ACTA | 56692358 | (see SEQ ID NO: 62) |
| SEQ ID NO: | R | CAATGCCACACTCACCTA | 56692087- | |
| 24 | | ACAC | 56692108 | |
| D13Mmu096 | | | | |
| SEQ ID NO: | F | AATAAGTTCTTGTTCCTTT | 96579927- | (GAAA)19 |
| 25 | | CCTC | 96579905 | (see SEQ ID NO: 63) |
| SEQ ID NO: | R | TTTGGCTGCCCTAAACTC | 96579792- | |
| 26 | | TTTGTAG | 96579816 | |
| D14Mmu074 | | | | |
| SEQ ID NO: | F | TCACTCCACCTTGAGAAC | 74031224- | (CTTT)17 |
| 27 | | TCT | 74031244 | (see SEQ ID NO: 64) |
| SEQ ID NO: | R | GCTCAGTGAACTCAGTC | 74031531- | |
| 28 | | CTAAG | 74031510 | |
| D15Mmu084 | | | | |
| SEQ ID NO: | F | CAAGGACACTGAGAGCA | 84867343- | (GAAA)2GAAG(GAAA)17 |
| 29 | | ACAGA | 84867364 | (see SEQ ID NO: 65) |
| SEQ ID NO: | R | GAAGAGGACTATGACTG | 84867847- | |
| 30 | | GCTGTT | 84867825 | |
| D16Mmu030 | | | | |
| SEQ ID NO: | F | CGCGACTGAAATGTCCT | 30299393- | (TAAA)7N(GAAA)16 |
| 31 | | GAATG | 30299372 | (see SEQ ID NO: 66) |
| SEQ ID NO: | R | ACATGAAGGCTTACAACC | 30299149- | |
| 32 | | ATCTGAA | 30299173 | |
| D17Mmu041 | | | | |
| SEQ ID NO: | F | TACAGCGGCAAAGAGAG | 41499189- | (AAGA)17 |
| 33 | | GAAG | 41499209 | (see SEQ ID NO: 67) |
| SEQ ID NO: | R | AAGAGAGACCCCTTGGT | 41499584- | |
| 34 | | ACTG | 41499564 | |
| D18Mmu069 | | | | |
| SEQ ID NO: | F | GAATGGATCTGCATCTGA | 69071032- | (CTTT)21 |
| 35 | | GGTT | 69071053 | (see SEQ ID NO: 68) |
| SEQ ID NO: | R | CTCGAACTTAGACAACTG | 69071314- | |
| 36 | | TGGTC | 69071314 | |
| D19Mmu008 | | | | |
| SEQ ID NO: | F | AGGCCTACAGACAGTTG | 8760764- | TTTC TTCC (TTTC)14 |
| 37 | | TGCA | 8760784 | (see SEQ ID NO: 69) |
| SEQ ID NO: | R | CTTCCATGCTAGGGCTA | 8761128- | |
| 38 | | GTGA | 8761108 | |
| DYMmu001 | | | | |
| SEQ ID NO: | F | CCCTTAATCAATTGCTGC | 334386- | (CTTT)21 |
| 39 | | AAA | 334406 | (see SEQ ID NO: 70) |
| SEQ ID NO: | R | AGCCACATGTGCTTGCTT | 334525- | |
| 40 | | TC | 334506 | |
| DYMmu002 | | | | |
| SEQ ID NO: | F | CTGAGTGATGCAGGGTG | 2565930- | (CTTT)20 |
| 41 | | C | 2565947 | (see SEQ ID NO: 71) |
| SEQ ID NO: | R | GGCTGTGAATCCAGAGG | 2566201- | |
| 42 | | CTT | 2566182 | |
| DYMmu003 | | | | |
| SEQ ID NO: | F | TGTAGTCCCCTGGATCC | 1728782- | (GAAA)8 |
| 43 | | CAT | 1728801 | GAGA |
| | | | | (GAAA)3 |
| | | | | (see SEQ ID NO: 72) |
| SEQ ID NO: | R | TGCCAGCTCAAGTTGTCT | 1729107- | |
| 44 | | TTTG | 1729128 | |

TABLE 2
Genomic DNA sequences of the inventive STR markers

| Marker | Sequence |
|---|---|
| Mouse | |
| D1Mmu121 | CAAAAGGAGGCGAGTAGGGTGACATTACTGTCCTAAAAATCAAGACT |
| SEQ ID NO: | CTTTCTTTCTTTCTTTCTTTCTTTCTTTCTTTCTTTCTTTCTTTCTTTCTT |
| 51 | TCTTTCTTTCTTTCTTTCTTTCTTTCTTTCTTTCTTTCTATCACAGAAAAA |
| | ATGCTCTCCCATTGTGCCACGTACTAT |
| D2Mmu008 | TAACAAGCAGCAATGATGAGTGCTTTGCAAATATATCTGGGCTATTCT |
| SEQ ID NO: | AAAATTGACTACTATAAACCACCCCATCTATCTAGACAGTTTTGTTTCC |
| 52 | TGGAGGGCATTCTGTTTTGTTCTGTAATGATATTTCAAGCTGTATGGG |
| | AGTCTTGTTTCCACACGTGACTACATTTATTAACTTAGCCACATCTAAA |
| | ATACAGTGTGGGAACCCAGTAAGGGTATATGGTATCTTAACTCTCATC |
| | CAATTTTCAGCTGGGAGCATTGCATGCTATTTTCCCCTTGTTTTCCTTT |
| | CTTTCTTTCTTTCTTTCTTTCTTTCTTTCTTTCTTTCTTTCTTTCTTTCTT |
| | TCTTTCTTTCTTTCTTTCTAATGTTTCTTTTCAAAGATTTTGAATTTGTA |
| | ATGAACACATGAGGGTATATATCCCACCTTGATCTAGCTGTGGAGATC |
| | CCCTTGGCTACTGTGGAAAATACCACTAGACAACTGTGGGAGATGTT |
| | GGCTGTAAGATCACTT |
| D3Mmu158 | GTGGCTCTTATCCAGAACCTAGGGAGGATTTAAGACATTTGGAAGGT |
| SEQ ID NO: | CCAAATGCTTAGCTGCAAGCATTGCACCCATTTCTCCCTTTCTTTCTTT |
| 53 | CTTTCTTTCTTTCTTTCTTTCTTTCTTTCTTTCTTTCTTTCTTTCTTTCTT |
| | TCTTTCTTTCTTTCTTTCTTTCTTTCTTTCTAATTTTTTATTAGGTATTTT |
| | CCTCATTTACATTTCTAATGCTATCCCAAAAGTCCCCCATACCCACCC |
| | CCCCCCACTCCCCTACCCACCAACTCTCCCTTTTTGGCCCTGGCGTT |
| | CCCCTGTACTGGGGCATATAAAGTTTTGCACCCATTTCTTATAAGGAA |
| | CTTCAGCATTTGTGGATTTTGGTTTGTGGTATACAGGATCTTGGAAGA |
| | ATCCCTTAGACACCAAGGAACTACTGACTACTGGACAGAGTATTTTGT |
| | ATGTGTAGTAGCTAGGTACCCAAGGCAGTGGATGTGTGGAGAAAGAA |
| D4Mmu155 | CAAACTGAGTGTGACGTAGGACAGGAATAAAGATCAGACCGCGAGG |
| SEQ ID NO: | CAAGGAGGCAAAAGAAAGAAAGAAAGAAAGAAAGAAAGAAAGAAAGA |
| 54 | AAGAAAGAAAGAAAGAAAGAAAGAAAGAAAGAAAGAAAGAAAGAAAA |
| | GGGAGAGAATCTGGTGGTTTTCTCTGTTGCTGCTGGGACT |
| D5Mmu108 | TGTGATACAAAGGCTGCCAGGCTTCAGTAGAGTATATTTGCCTTCCTG |
| SEQ ID NO: | GGTGCCTATTCTGTTTTCCTTTCTTTCTTTCTTTCTTTCTTTCTTTCTTT |
| 55 | CTTTCTTTCTTTCTTTCTTTCTTTCTTTCTTTCTTTCTTTCTTTCTCTGAG |
| | ACAGGGTTTCTCTGTGTAGCCCTGGCTGTCCTGGAACTCGCTTTGTA |
| | GACCAGGCTGGCCTCGAACTCAGTAATCCACCTGCCTCTGCCTCCCA |
| | AATGCTGGGATTAAAGGCGATAACCCCTTTCAACCTCATTCTTTGCAT |
| | GCCCCTTCTCCCAAACACCTGCTGGT |
| D6Mmu120 | TCTGGAGCTCAAAGAAACATAGAGTTATTGCAATTAATTTCTTTCTTTC |
| SEQ ID NO: | TTTCTTTCTTTCTTTCTTTCTTTCTTTCTTTCTTTCTTTCTTTCTTTCGAG |
| 56 | AGACAGGTCTTACTCTGTCTTACTATTTAGCCCTG |
| D7Mmu003 | CTTGGACCTTCATATTCAGTGTGGGAAAGAAAGAAAGAAAGAAAGAAA |
| SEQ ID NO: | GAAAGAAAGAAAGAAAGAAAGAAAGAAAGAAAGAAAGAAAGAAAGAA |
| 57 | AGAAAGAAAGAAAGAAAGGTAAAAAAGAGGGAGGATTTGAAACTCTG |
| | GA |
| D8Mmu127 | CTAGACAGGGGATCTGGCTAGGAGGAGTGTTTGCAGAAGAAACAGG |
| SEQ ID NO: | CATTAACTCTTAACTCTTGCCCAGGCCACTCTGTTTCTAGCTGGGATG |
| 58 | ACACCATCAGAATTCTTTCTTTCTTTCTTTCTTTCTTTCTTTCTTTCTTT |
| | CTTTCTTTCTTTCTTTCTTTCTTTCTTTCTTTCTTTCTTTCTTCTTCTTTT |
| | GGACACAGTTTCAGAAGGGGG |
| D9Mmu100 | TAGATCTGTGCACTAAGTCCGACACACAAATAAAATACAGAACGAAAA |
| SEQ ID NO: | GCCCAGAGGAGAGAAAGAATCCTCAAGAAACATCCAAGAAGACATTG |
| 59 | CAGAGTGTGAAGAGTGAAGGGCTCTAGCCCAGGCCCACATCATGGT |
| | GTATCTAGGGAAAGTTTTATGAGCTTCTAGAAAGAAAACAAACACAAT |
| | CAAGACATACATTTATTTGTTTATTCTTTCTTTCTTTCTCTCTCCTTCTT |
| | TCTTTCTTTCTTTCTTTCTTTCTTTCTTTCTTTCTTTCTTTCTTTCTTTCT |
| | TTCTTTCTTTCTTTCTTTCTTCATTAATTCATTCATTTATGTGTGTGGCT |
| | GTCTTGCCTGCATGGTTATCTTTATACCACATGTGCACTGACTGCTCT |
| | CAGATGTCAGA |
| D10Mmu043 | AGGGCCAGCCTCTGCTATGTCGGGAATTTTATGAGACCTTGTCAGAA |
| SEQ ID NO: | AGAAAGAAAGAAAGAAAGAAAGAAAGAAAGAAAGAAAGAAAGAAAGA |
| 60 | AAGAAAGAAAGAAAGAAAGAAAGAAAGAAAGGGAAAGAAAGAAACCA |
| | TAGGAAGTAATTTGTGCATTAAAATGTGTGTTAAGTGAATGTAGTCATT |
| | TATCCAACCCATATTTAGGTCTCATTTATAAAAACAGTGACCCAGAATA |
| | TGCATCTAACGTGAGTAGCCTGAAGCCGAAGCGCCTGTGTACAGAGT |
| | GGGTTGAGAGAGGATACTTGGTGATCTACGTACTCATTTCTAGC |
| D11Mmu030 | CAGTTGCTGATAACCTGACAGCCAAAATTCCACAGAGCCACATAACA |
| SEQ ID NO: | GAAGAAGTGAACATACATTTACATGCATAGTCACTTAACACACTTAAAT |
| 61 | AGAAAAGAAAGAAAGAAAGAAAGAAAGAAAGAAAGAAAGAAAGAAAG |
| | AAAGAAAGAAAGAAAGAAAGAAAGAAAGAAAGAAAGGAAATAGAGAA |
| | AAGAGATTGCCAATATGAAAGAAGTGACTCTAGCCCATTCAAAACTGT |
| | ATAAAAGAATGTCTGGAGCCTGGCTGGGGGAATGGATACCAATCATG |
| | CCAACATCCTAGAAACCTTTGCTGGAGCCACTGACACACCCACCAAG |
| | AAACAAGCTTCTTACTGGGTGCTTCTGGCCTGAGGCATAAACATTGGT |
| | CAGGAACACTGGG |
| D12Mmu056 | CAATGCCACACTCACCTAACACAGGCGTGCTGTAGAGGAACTCAACT |
| SEQ ID NO: | CGAGGTCCAGCCTTTGCCTGAAAACTTCCCCCAGACAGCTGCAAGAA |
| 62 | CTCACAGTACTGGTTTTTGTGTGTCTTTCTTTCTTTTCTTTCTTTCTTTC |
| | TTTCTTTCTTTCTTTCTTTCTTTCTTTCTTTCTTTCTTTCTTTCTTTCTTT |
| | CCTTCTTTCTTTTTCTTATCCATGTCCAGGAGCTGTCCTGGACACTGG |
| | CTGCTTCCCAAGGAGCTTCGGTAGGGCATAGTGACCTGGACCTTGCT |
| | TG |
| D13Mmu096 | TTTGGCTGCCCTAAACTCTTTGTAGTGCCCAATTGAAGAAAGAAAGAA |
| SEQ ID NO: | AGAAAGAAAGAAAGAAAGAAAGAAAGAAAGAAAGAAAGAAAGAAAGA |
| 63 | AAGAAAGAAAGAAAGAAAGAGGAAAGGAACAAGAACTTATT |
| D14Mmu074 | TCACTCCACCTTGAGAACTCTCTTTGTGCCTATTAGATTTTTCATTTTA |
| SEQ ID NO: | GATCTTTCTTCATTTTTATACCAGTGTTGTGGTTGAGTTTTGTTTCTATT |
| 64 | GTTTTTTCCATGAATTTGACCAAGAAAAGCCCTGGGAAAGAATTTCTT |
| | TCTTTCTTTCTTTCTTTCTTTCTTTCTTTCTTTCTTTCTTTCTTTCTTTCT |
| | TTCTTTCTTTCTTTTTTTTTTTTAAGCTAGCTGCATACACTTGCATATCT |
| | TGCCCTAGGCTGTGGGACAATAAACTTACTTTTCTAACCTTAGGACTG |
| | AGTTCACTGAGC |
| D15Mmu084 | CAAGGACACTGAGAGCAACAGAGAGGCCCTCTGTTGCTGAGGAGTC |
| SEQ ID NO: | TCTATCCCAACTCAGCCCCCAACACTGAGCATCTCCCTCACAATTTCC |
| 65 | ATCCCAGACCCCCATAATAACAGGAGGGGCCTAGGGAGCCCTTCCTA |
| | CTCTCTTGAATACCACCAATAAAGTTCGCTGCACAGAAAGAAAGAAGG |
| | AAAGAAAGAAAGAAAGAAAGAAAGAAAGAAAGAAAGAAAGAAAGAAA |
| | GAAAGAAAGAAAGAAAGAAAGAGCTAACCTCTGGTATTTCCCCAGAC |
| | TGAACTCTCAGAGTTGGAAGAGACTCCCAAGGTCACATTGCCCATTC |
| | TCTTACTTTATGGATAAGAAAGGGTGGTTCAGAGAACTTGAAATAGCC |
| | CTCAGGCAGTGCGTGGCAGGAGGATTTTGACCTCAACCTGATCTTTT |
| | CTCCATAATCAGTGGGCAGTGATCTACCACCACCTTCTGCTTGTGAG |
| | GCAGACATGAAACAGCCAGTCATAGTCCTCTTC |
| D16Mmu030 | ACATGAAGGCTTACAACCATCTGAACAGCTACAGTATACTCACATACA |
| SEQ ID NO: | ATAAATAAATAAATAAATAAATAAATAAACAAATAAATAAATCTTTTAAG |
| 66 | AAAGAAAGAGAGAGAGGAAGAAAGGAAGAAAGAAAGAAAGAAAGAAA |
| | GAAAGAAAGAAAGAAAGAAAGAAAGAAAGAAAGAAAGAAAGAAACAG |
| | GATTTGTATCTAGAAATGCCTACATGAGTTGCATTCAGGACATTTCAG |
| | TCGCG |
| D17Mmu041 | TACAGCGGCAAAGAGAGGAAGAAAGAAAGAAAGAAAGAAAGAAAGAA |
| SEQ ID NO: | AGAAAGAAAGAAAGAAAGAAAGAAAGAAAGAAAGAAAGACAGACAAA |
| 67 | TGCAGATTTCTAAAATTTAGGAGTAACTGAAGTATATTTTTAAAATGCA |
| | AAATTGGTCCTGTTTTTATGGGAACACTAATGGCAACTCAACCCCAGA |
| | TAATTTTTCTTATTTATTTTATCTATCTATCTATCTATTTATTTATTTATTT |
| | ATTTATTACTAGATATTTTCTTTATATACATTTCAAATGCTATCCTGAAA |
| | GTTCCCTATACTCTCCCTCTGCCCTGCTCCCCTAACCACCCACTCCC |
| | GCTTCTTGGCCCTGTACTGGGGCATATAAAGTTTGCAGTACCAAGGG |
| | GTCTCTCTT |
| D18Mmu069 | GAATGGATCTGCATCTGAGGTTTTCTTCCTTCCTCCCTCCCTCCCTCC |
| SEQ ID NO: | CTTGCTTCTTTCTTTCTTTCTTTCTTTCTTTCTTTCTTTCTTTCTTTCTTT |
| 68 | CTTTCTTTCTTTCTTTCTTTCTTTCTTTCTTTCTTTCTTTCCTTCATGTAT |
| | CCTGTCTGTTTTTAAGATTAGCCACATAGAGAGCAATTTGATAACAGA |
| | TTGGAGTACTTCTGTGAGTCTGCCCTCCAGCTGACTAAAACCCTGATT |
| | GCTAGGAAAGAATGGACCACAGTTGTCTAAGTTCGAG |
| D19Mmu008 | AGGCCTACAGACAGTTGTGCAGTATAGTGGGTGCTGAGAATCAAAGC |
| SEQ ID NO: | TGGTCCTCTAGAAGAGCAGCTAGCTTGTTTTCTTCCTTTCTTTCTTTCT |
| 69 | TTCTTTCTTTCTTTCTTTCTTTCTTTCTTTCTTTCTTTCTTTCCTGTAAGT |
| | ACACTGTAGCTGTTTTCAGACACTCCAGAGGAGGGCGCCAGATCTCA |
| | TTACAGATGGTTGTGAGCAACAGTGTGGTTGCTGGGAATTGAACTCA |
| | GAATCTTCAGAAGAGCAAGTTAGTGCTCTTAACCACTCTGCCATCTCT |
| | CCAGCCCCCCATCCCCAACTAGCTAGTTTTCTTGAGAACATTATGACT |
| | AGCCCAATCACTAGCCCTAGCATGGAAG |
| DYMmu001 | AGCCACATGTGCTTGCTTTCTTTCTTTCTTTCTTTCTTTCTTTCTTTCTT |
| SEQ ID NO: | TCTTTCTTTCTTTCTTTCTTTCTTTCTTTCTTTCTTTCTTTCTTTCTTTAT |
| 70 | CAGAAACAAGAGTATCCATTTGCAGCAATTGATTAAGGG |
| DYMmu002 | CTGAGTGATGCAGGGTGCTACTCTGCATCTGTGCCAGAATTGCAGAA |
| SEQ ID NO: | GGTTTTCTTTCTTTCTTTCTTTCTTTCTTTCTTTCTTTCTTTCTTTCTTTC |
| 71 | TTTCTTTCTTTCTTTCTTTCTTTCTTTCTTTCTTTTAACTGTTGGGAGTG |
| | CCCACTTGGCTTAGTGATTCAAAACAGGTCCTGAACTACAGGAAATCA |
| | GCTCTTCCTTGAATGAGCTTGCATAGAAGGAGGACCCTGATCCTAAC |
| | TGATAAGAAAAGCCTCTGGATTCACAGCC |
| DYMmu003 | TGTAGTCCCCTGGATCCCATAGTCTAGAGTGACACATAAATATGAAAA |
| SEQ ID NO: | TGTTTATGCAAAAATGCACAGAGGAGAGAAAGGAAACAATGAGAAATA |
| 72 | GAAAGAGAGAGAGATGGAGAAAGATAGAGACAGAGAGACAAATATAG |
| | TGAAAGAGAGAAAGAAAGAAAGAAAGAAAGAAAGAAAGAAAGAGAGA |
| | AAGAAAGAAAGAGCACAATAGGATGCAGAAGAAATGGATTGAGCAAT |
| | ATTGTCTCAGAGTATAAGGAACAAAGAACAGGATGAAGGATAATGAC |
| | CCAAGAGACAAAAGACTTATCAATTCACCACACTGAATGGTCAAAAGA |
| | CAACTTGAGCTGGCA |
| Rat | |
| DYRno004 | GTCAGAAGGGGCAGACTCTAAAACCTTCAAAGCTATGCACTGTAGAC |
| SEQ ID NO: | TTTTGAATACTGCTTTCTAAGGACATCTTAACAGAAATGCGGTTTGTTT |
| 73 | ATTGAATTTATTCTTTCCTTCCTTCCTTCTTTCTTTCTTTCTTTCTTTCTT |
| | TCTTTCTTTCTTTCTTTCTTTCTTTCTTTCTTTCTTTCTTTCTTTCTTTTT |
| | ATACTCCAGATTTAATTCCCTTCCAGTTCACCATCCCAAGTGA |
| DYRno004a | TCACTTGGGATGGTGAACTGGAAGGGAATTAAATCTGGAGTATAAAAA |
| SEQ ID NO: | GAAAGAAAGAAAGAAAGAAAGAAAGAAAGAAAGAAAGAAAGAAAGAA |
| 74 | AGAAAGAAAGAAAGAAAGAAAGAAGGAAGGAAGGAAGGAAAGAATAA |
| | ATTCAATAAACAAACCGCATTTCTGTTAAGATGTCCTTAGAAAGCAGT |
| | ATTCAAAAGTCTACAGTGCATAGCTTTGAAGGTTTTAGAGTCTGCCCC |
| | TTCTGACT |
| DYRno165 | CAGACCTCAGGGCATTTCCATATTCTAAGTTAGCAACTATACAAGTTA |
| SEQ ID NO: | TGAGGACTAGTACAAAATGCCCAGCATCTATGTATCTATGTATCTATG |
| 75 | TATCTATGTATCTATGTATCTATGTATCTATGTATCTTCTATCTATCTAT |
| | CTATCTATCTATCTATCTATCTATCTATCTATCTATCTATCTATCTATCA |
| | ATCATCTAAGAAACCAGAGAACAAAGAGCCATAT |
| DYRno304 | CAGATCTCAGGGCATTCCCATATTCTAAGTCAACAACTCTTCATGTTA |
| SEQ ID NO: | TGAAGACTAGTATGAAATCCCCAGCATCTATCTATCTATCTATCTATCT |
| 76 | ATCTATCTATCTATCTATCTATCTATCTATCTATCTATCTATCGATCTAT |
| | CTAAGAAACCAGAAAACAAAGAGCCATAT |

The invention is more particularly elucidated below on the basis of 1 drawing and 6 exemplary embodiments.

What is shown by:

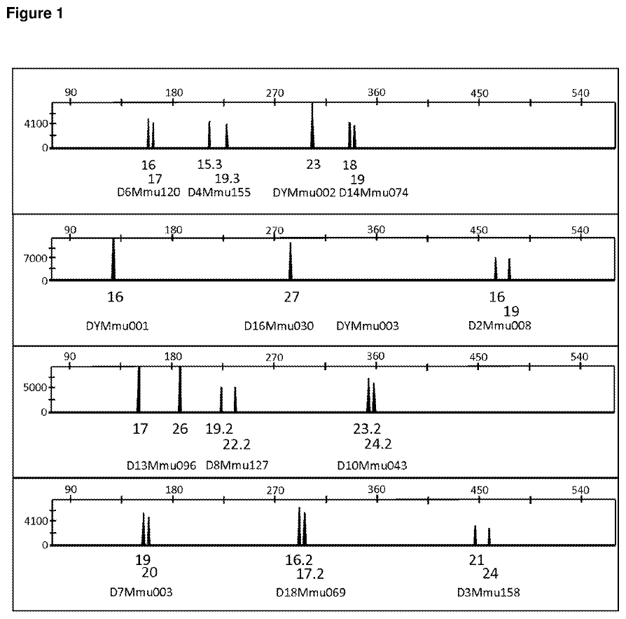

FIG. 1 is an electropherogram which depicts the coamplification of 11 autosomal STR markers and of the Y-chromosomal markers DYMmu001, DYMmu002 and DYMmu003 of a DNA sample of the type M. m. domesticus. The PCR products of the STR markers are labeled with the following fluorescent dyes and sorted in order of increasing size within a dye: dye 6FAM™: D6Mmu120, D4Mmu155, DYMmu002, D14Mmu074; dye DY530™: DYMmu001, D16Mmu030, DYMmu003, D2Mmu008; dye ATTO550™: D13Mmu096, D8Mmu127, D10Mmu043; dye DY510XL™: D7Mmu003, D18Mmu069, D3Mmu158. The size standard used to calculate the fragment lengths is labeled with the dye DY632™.

### EXEMPLARY EMBODIMENT 1: ASSIGNMENT OF THE Y CHROMOSOMES IN SELECTED OUTBRED POPULATIONS TO THE TYPES M. M. MUSCULUS AND M. M. DOMESTICUS

Describing the genetic diversity of an outbred population requires knowledge of the number of different variants of the Y chromosome in the population. According to the invention, this object is achieved by first performing an assignment of the Y chromosomes of outbred populations to the two types M. m. domesticus and M. m. musculus. This requires markers which can assign a Y chromosome to one of the two basic variants. Surprisingly, it was possible to identify the STR marker DYMmu003, which has different alleles in different variants of M. m. musculus. By contrast, the PCR primers used do not have a homologous sequence for all variants of the Y chromosome of the type M. m. domesticus. The result of this is that, in the case of the presence of a chromosome of the type M. m. domesticus, no PCR product is generated. DYMmu003 is utilized to establish the belonging of Y chromosomes to one of the two types. To this end, between 5 and 25 male individuals of the outbred populations Hsd:ICR(CD1), Crl:CD1(ICR), RjOrl:SWISS, Crl:NMRI, Han:NMRI, RjHan:NMRI and HsdWin:NMRI were analyzed in each case. Altogether 100 male animals of 7 different outbred populations of mouse were analyzed using the STR marker DYMmu003 and assigned accordingly to M. m. musculus or M. m. domesticus (Table 3). In the case of type M. m. domesticus, DNA products are not obtained for either of the two markers; in the case of type M. m. musculus, alleles are always detected. Completely unexpectedly, only the outbred population Crl:NMRI has Y chromosomes of both types, whereas the outbred populations Hsd:ICR(CD1), Crl:CD1(ICR), RjOrl:SWISS and RjHan:NMRI have solely variants of the type M. m. domesticus and Han:NMRI and HsdWin:NMRI can always be assigned only to the type M. m. musculus.

TABLE 3
Assignment of Y chromosomes to the type M. m. domesticus
and M. m. musculus in various outbred populations
(N = number of tested individuals)

| | | M. m. | M. m. |
|---|---|---|---|
| | N | domesticus | musculus |
| Hsd:ICR(CD1) | 25 | 25 | 0 |
| Crl:CD1(ICR) | 5 | 5 | 0 |
| RjOrl:SWISS | 20 | 20 | 0 |
| Crl:NMRI | 20 | 17 | 3 |
| Han:NMRI | 20 | 0 | 20 |
| RjHan:NMRI | 5 | 5 | 0 |
| HsdWin:NMRI | 5 | 0 | 5 |

### EXEMPLARY EMBODIMENT 2: ASSIGNMENT OF ALLELES OF THE STR MARKERS DYMMU001, DYMMU002 AND DYMMU003 OF THE Y CHROMOSOME IN SELECTED OUTBRED POPULATIONS

It is further an object of the present invention to make available STR markers for the Y chromosome that are polymorphic both in the case of the type M. m. musculus and in the case of the type M. m. domesticus and can be used for describing Y-chromosomal haplotypes. The inventive STR markers DYMmu001 and DYMmu002 have this property. On the basis of DNA samples from male animals of various inbred strains and outbred populations of mouse, the possible allele spectrum for the markers DYMmu001, DYMmu002 and DYM003 was determined.

Altogether 50 male animals of 15 different inbred strains and 85 male animals of 7 different outbred populations of mouse were analyzed using the markers DYMmu001, DYMmu002 and DYMmu003 (Table 4). The assignment to M. m. musculus or M. m. domesticus in the case of the outbred populations was done on the basis of the relevant investigation results for DYMmu003. Some alleles were detected solely in inbred strains.

TABLE 4
Detected alleles of the STR markers DYMmu001, DYMmu002
and DYMmu003 and the assignment thereof in outbred populations
to M. m. musculus and M. m. domesticus

| | | Outbred | |
|---|---|---|---|
| | Inbred + outbred | M. m. | M. m. |
| | Alleles found | domesticus | musculus |
| DYMmu001 | 16, 17, 18, 19, 21, | 16, 17, 18, 20, | 21, 22, 23 |
| | 20, 22, 23, 24, 25, 26 | 21 | |
| DYMmu002 | 17, 19, 20, 21, 23, | 17, 23, 24, 25 | 19, 20 |
| | 24, 25 | | |
| DYMmu003 | 12, 14 | No PCR product | 14 |

### EXEMPLARY EMBODIMENT 3: DETECTION OF DIFFERENT Y-CHROMOSOMAL HAPLOTYPES WITHIN OUTBRED POPULATIONS OF MOUSE

Since the Y chromosome does not recombine, the analysis results of different Y-STR markers can be combined with one another and presented in the form of haplotypes. Table 5 presents different Y-chromosomal haplotypes for 7 different outbred populations of CD1, SWISS and NMRI, which were obtained on the basis of the analysis results for the STR markers DYMmu001, DYMmu002 and DYMmu003. With the exception of the outbred population HsdWin:NMRI with 5 identical Y-STR haplotypes among 5 tested individuals, all the other outbred populations have more than one haplotype. According to the invention, it is possible to perform, within a specific outbred population, an assignment of male individuals to different haplotypes and also the assignment to one of the two basic Y-chromosome types M. m. musculus or M. m. domesticus. This approach to describing the genetic diversity of outbred populations via the Y chromosome is novel and can be used in future for genetic monitoring.

Altogether 100 male animals of 7 different outbred populations of mouse were analyzed using the STR markers DYMmu001, DYMmu002 and DYMmu003. The haplotypes were derived from the results of the analysis of the single samples, the haplotype results being presented in the order DYMmu001-DYMmu002-DYMmu003. The value “0” for DYMmu003 indicates that no PCR product is detectable and that a chromosome of the type M. m. domesticus is concerned. According to the invention, it is possible to detect different combinations of alleles within an outbred population, and this demonstrates the presence of various Y chromosomes within an outbred population that are distinguishable from one another.

The comparison of haplotypes between various outbred populations shows that there are in some cases haplotypes which are unique for a specific outbred population and were not able to be detected in another outbred population.

TABLE 5
Y-chromosomal haplotypes in various outbred populations. The
order of haplotype indication is DYMmu001-DYMmu002-DYMmu003

| Hsd:ICR | Crl:CD1 | | | | | |
|---|---|---|---|---|---|---|
| (CD1) | (ICR) | RjOrl:SWISS | Crl:NMRI | Han:NMRI | RjHan:NMRI | HsdWin:NMRI |
| N = 25 | N = 5 | N = 20 | N = 20 | N = 20 | N = 5 | N = 5 |
| 16-23-0 | 17-24-0 | 16-23-0 | 19-17-0 | 21-19-14 | 17-24-0 | 23-20-14 |
| 16-24-0 | 17-25-0 | 17-23-0 | 20-17-0 | 21-20-14 | 17-25-0 | |
| 17-23-0 | 18-24-0 | 17-24-0 | 21-17-0 | | | |
| 17-24-0 | | | 22-20-14 | | | |
| 17-25-0 | | | | | | |

### EXEMPLARY EMBODIMENT 4: CHARACTERIZATION OF THE RAT Y CHROMOSOME WITH THE AID OF STR MARKERS

Male animals of 17 different strains of Rattus norwegicus were analyzed using the inventive Y-chromosomal markers DYRno004, DYRno165 and DYRno304. The alleles were designated on the basis of the measured lengths of the amplification products. This is sufficient for documenting the diversity of different alleles. Exact knowledge of the DNA sequences of the individual alleles is not absolutely necessary for this purpose. The haplotypes derived from the analysis results are presented in Table 7 in the order DYRno004, DYRno165 and DYRno304. All the strains are distinguishable from one another. These markers are thus suitable for genotyping the rat Y chromosome and for establishing the genetic diversity within outbred populations of rat. Table 6 brings together the inventive STR markers, the oligonucleotide primers used, the chromosomal location thereof and the structure of the repeat units. For each primer, what is specified is the chromosomal location thereof in base pairs, which was ascertained on the basis of reference DNA (NCBI, version Rnor_6.0). Since the rat Y chromosome is only very short, a detailed assignment was performed to characterize the chromosomal position of the marker. DYRno165 corresponds to the position at 1.65 megabases.

The oligonucleotide primer pair of the marker DYRno004 binds specifically to two different target regions of the Y chromosome and an allele product is obtained for each target region. The two alleles amplified in this connection usually differ from one another with respect to the number of repeat units, meaning that two different alleles can be detected simultaneously using one primer pair. Assigning the individual alleles to the specific target region is, nevertheless, not possible. The STR marker DYRno004 is thus a double marker which is highly polymorphic and is ideally suitable for creating a Y-specific haplotype. The smaller of the two alleles is listed first in the haplotype, then the second, larger allele. For the two markers DYRno165 and DYRno304, the same reverse primer is used; owing to the different forward primer, it is possible to perform an unambiguous assignment of alleles to the specific marker. According to the invention, the two specific forward primers can be provided with different fluorescent dyes, and this allows an unambiguous assignment of the amplification products.

TABLE 6
List of the inventive Y-chromosomal STR markers in the case of rat Rattus
norwegicus, the oligonucleotide primers used, the location thereof on the Y
chromosome in base pairs and the pattern of the STR repeat unit. The number of
repeat units is based on the reference sequence deposited in the NCBI database
(version Rnor_6.0). The alleles were named according to the guidelines (DNA
Recommendations, 1997). (F-forward primer, R-reverse primer)

| | | | Y-chromosomal | |
|---|---|---|---|---|
| | | | position | |
| Marker | | Primer sequence (5′-3′) | (bp) | Repeat unit |
| DYRno004 | | | | |
| SEQ ID NO: | F | GTCAGAAGGGGCAGACT | 49153-49172 | CTTT(CCTT)3 |
| 45 | | CTA | 249767- | (CTTT)18 |
| | | | 249748 | (see SEQ ID NO: 73 |
| SEQ ID NO: | R | TCACTTGGGATGGTGAA | 49393-49374 | and 74) |
| 46 | | CTG | 249523- | |
| | | | 249542 | |
| DYRno165 | | | | |
| SEQ ID NO: | F | CAGACCTCAGGGCATTT | 1657537- | (ATCTATGT)7ATCT |
| 47 | | CCAT | 1657557 | (TCTA)15 |
| SEQ ID NO: | R | ATATGGCTCTTTGTTCTC | 1657766- | (see SEQ ID NO: 75) |
| 48 | | TGG | 1657746 | |
| DYRno304 | | | | |
| SEQ ID NO: | F | CAGATCTCAGGGCATTC | 3047184- | (ATCT) 16 |
| 49 | | CCA | 3047203 | (see SEQ ID NO: 76) |
| SEQ ID NO: | R | ATATGGCTCTTTGTTCTC | 3047359- | |
| 50 | | TGG | 3047339 | |

Table 7 brings together the analysis results of the genotyping of 17 different strains of Rattus norwegicus using the STR markers DYRno004, DYRno165 and DYRno304. The designation of the alleles corresponds to the measured lengths of the amplification products. In principle, this is sufficient for demonstrating the diversity of different alleles without specific knowledge of the underlying DNA sequence. The presented haplotypes are presented in the order of the analysis results for DYRno004, DYRno165 and DYRno304. Each strain has its own haplotype. The combination of the three inventive STR markers is suitable for representing the genetic diversity of the Y chromosome in the case of rat.

TABLE 7
Y-chromosomal haplotypes in various strains of rat

| | STR loci | | | |
|---|---|---|---|---|
| Rat strain | DYRno004 | DYRno165 | DYRno304 | Haplotype |
| ACI | 255-258 | 243 | 188 | 255-258-243-188 |
| BN | 254-264 | 231 | 184 | 254-264-231-184 |
| BS | 259-264 | 243 | 192 | 259-264-243-192 |
| DA | 234-247 | 247 | 192 | 234-247-247-192 |
| E3 | 259-267 | 247 | 192 | 259-267-247-196 |
| F344 | 255-267 | 243 | 196 | 255-267-243-196 |
| LE | 259-267 | 247 | 196 | 259-267-247-196 |
| LEW | 259-264 | 251 | 188 | 259-264-251-188 |
| LOU-C | 264-267 | 247 | 192 | 264-267-247-192 |
| MNS | 255-271 | 247 | 192 | 255-271-247-192 |
| NAR | 255-259 | 247 | 192 | 255-259-247-192 |
| OM | 251-254 | 231 | 184 | 251-267-231-184 |
| PAR | 251-267 | 243 | 196 | 251-267-243-196 |
| PVG | 251-264 | 251 | 211 | 251-264-251-211 |
| WC | 264-275 | 239 | 196 | 264-275-239-196 |
| WF | 255-255 | 247 | 192 | 255-255-247-192 |
| WKY | 259-283 | 255 | 188 | 259-283-255-188 |

### EXEMPLARY EMBODIMENT 5: DETERMINATION OF THE GENOTYPE FOR ANIMALS OF THE OUTBRED POPULATIONS HSD:ICR(CD1) AND RJORL:SWISS

The genotypes were determined for 20 male animals in each case of the outbred populations Hsd:ICR(CD1) and RjOrl:SWISS, and the PIC and Het values were calculated on the basis thereof. It is essential for the assessment of the quality of a marker that multiple different alleles are detectable and that none of the alleles should represent more than 50% of the alleles in a population. To be able to estimate this, the analysis of 20 animals per population was deemed sufficient. In the case of such a number of animals, there are results across altogether 40 alleles (2 per animal); an allele with a proportion of 10% in the population is, in this connection, detected four times from a statistical point of view.

The number of possible reference alleles as ascertained following the genotyping of a multiplicity of inbred strains and outbred populations varies, depending on the marker, between 7 and 11. For Hsd:ICR(CD1) and RjOrl:SWISS, STR markers were identified in which it was possible to detect the presence of at least 6 to 9 different alleles among the 20 genotyped animals. The PIC values vary between 0.34 and 0.84. The heterozygosity rate is between 0.50 and 0.95.

TABLE 8
Characterization of STR markers in RjOrl:SWISS and Hsd:ICR(CD1)
on the basis of the number of detected alleles per marker (No.)
and the biostatistical parameters “polymorphism information
content” (PIC) and heterozygosity (Het). The column “Refer.
allele” presents the total number of alleles which were observed
altogether in 150 individuals of various inbred and outbred strains
and substrains of inbred strains of mouse. The column “No.”
indicates how many different alleles were detected among the 20
DNA samples of male individuals of the specific outbred population.

| | Refer. | RjOrl:SWISS | | | Hsd:ICR(CD1) | | |
|---|---|---|---|---|---|---|---|
| STR locus | allele | No. | PIC | Het | No. | PIC | Het |
| D1Mmu121 | 10 | 8 | 0.64 | 0.75 | 6 | 0.70 | 0.85 |
| D2Mmu008 | 7 | 6 | 0.67 | 0.65 | 6 | 0.75 | 0.75 |
| D3Mmu158 | 8 | 7 | 0.79 | 0.90 | 6 | 0.69 | 0.60 |
| D4Mmu155 | 8 | 6 | 0.72 | 80.0 | 6 | 0.69 | 0.75 |
| D5Mmu108 | 8 | 6 | 0.63 | 70.0 | 6 | 0.71 | 0.95 |
| D6Mmu120 | 7 | 7 | 0.75 | 0.85 | 7 | 0.79 | 0.80 |
| D7Mmu003 | 9 | 6 | 0.72 | 0.70 | 7 | 0.75 | 0.85 |
| D8Mmu127 | 10 | 7 | 0.74 | 0.75 | 7 | 0.63 | 0.65 |
| D9Mmu100 | 10 | 5 | 0.50 | 0.45 | 8 | 0.83 | 0.90 |
| D10Mmu043 | 7 | 7 | 0.80 | 0.75 | 6 | 0.73 | 0.70 |
| D11Mmu030 | 11 | 6 | 0.71 | 0.85 | 4 | 0.57 | 0.75 |
| D12Mmu056 | 8 | 7 | 0.78 | 0.80 | 4 | 0.60 | 0.70 |
| D13Mmu096 | 11 | 9 | 0.84 | 0.90 | 7 | 0.79 | 0.85 |
| D14Mmu074 | 8 | 6 | 0.72 | 0.85 | 4 | 0.64 | 0.75 |
| D15Mmu084 | 9 | 9 | 0.73 | 0.65 | 5 | 0.52 | 0.50 |
| D16Mmu030 | 10 | 6 | 0.72 | 0.85 | 5 | 0.71 | 0.75 |
| D17Mmu041 | 7 | 5 | 0.61 | 0.75 | 3 | 0.34 | 0.55 |
| D18Mmu069 | 11 | 7 | 0.78 | 0.65 | 7 | 0.67 | 0.70 |
| D19Mmu008 | 8 | 7 | 0.72 | 0.70 | 5 | 0.51 | 0.65 |

### EXEMPLARY EMBODIMENT 6: ELECTROPHEROGRAM OF A MULTIPLEX PCR OF 14 STR LOCI

FIG. 1 depicts an electropherogram of a multiplex PCR of 14 STR loci, in which the DNA sample of a male animal of Hsd:ICR(CD1) was analyzed. One primer in each case of an oligonucleotide pair was covalently coupled to the following dye: dye 6FAM™: D6Mmu120, D4Mmu155, DYMmu002, D14Mmu074; dye DY530™: DYMmu001, D16Mmu030, DYMmu003, D2Mmu008; dye ATTO550™: D13Mmu096, D8Mmu127, D10Mmu043; dye DY510XL™: D7Mmu003, D18Mmu069, D3Mmu158. The size standard used to calculate the fragment lengths is labeled with the dye DY632™.

The PCR was carried out in a total volume of 25 μl using Snooplex® FastPrep PCR reagents (GVG Genetic Monitoring, Leipzig). The reaction mix for the PCR analysis consisted of the following components: 2.1 μl of nuclease-free water, 5 μl of 5×PCR buffer, 0.8 μl of Taq DNA polymerase, primer mixtures of the STR markers D2Mmu008 (0.8 μl), D3Mmu158 (1.2 μl), D4Mmu155 (0.8 μl), D6Mmu120 (1.6 μl), D7Mmu003 (1.2 μl), D8Mmu127 (1.2 μl), D10Mmu043 (0.4 μl), D13Mmu096 (0.7 μl), D14Mmu074 (1.6 μl), D16Mmu030 (1.6 μl), D18Mmu069 (1.2 μl), DYMmu001 (1.0 μl), DYMmu002 (0.8 μl), DYMmu003 (1.0 μl) and 2 μl of DNA.

The inventive multiplex PCR shown in exemplary embodiment 6 does not lead to any detectable alleles when using DNA of human origin or of rat, hamster or zebrafish.

### REFERENCES

Rapp K G (1972) HAN-rotation, a new system for rigorous outbreeding. Z Versuchstierkunde [Journal of laboratory animal science] 14: 133-142
Pertile M D, Graham A N, Choo K H, Kalitsis P (2009) Rapid evolution of mouse Y centromere repeat DNA belies recent sequence stability. Genome Res 19(12): 2202-2213
Nelson V R, Spezio S H, Nadeau J H (2010) Transgenerational genetic effects of the paternal Y chromosome on daughters' phenotypes. Epigenomics 2(4): 513-521
Kluge R, Wedekind D (2016) Auszuchtstāmme. Zuchtmethoden und genetische Eigenschaften [Outbred strains. Breeding methods and genetic properties]. Fachinformation aus dem Ausschuss für Genetik und Labortierzucht [Technical information from the committee for genetics and laboratory-animal breeding]. GV-SOLAS
Yalcin B, Nicod J, Bhomra A, Davidson S, Cleak J, Farinelli L, Osteras M, Whitley A, Yuan W, Gan X (2010) Commercially available outbred mice for genome-wide association studies. PLoS Genet 6(9). pii: e1001085
Witmer P D, Doheny K F, Adams M K, Boehm C D, Dizon J S, Goldstein J L, Templeton T M, Wheaton A M, Dong P N, Pugh E W, Nussbaum R L, Hunter K, Kelmenson J A, Rowe L B, Brownstein M J (2003) The development of a highly informative mouse simple sequence length polymorphism (SSLP) marker set and construction of a mouse family tree using parsimony analysis. Genome Research 13: 485-491
WO 2018/215436 PCT/EP2018/063343
WO 2016/008894
Shang H T, Wei H, Yue B F, Xu P (2008) Microsatellite analysis in Wistar and Spague-Darley ourbred rats. Fen Zi Xi Bao Sheng Wu Xue Bao. 41(1): 28-34. In Chinese, abstract in English
Clapcote S J and Roder J C (2006) Deletion polymorphism of Disc1 is common to all 129 Mouse substrains: Implications for gene targeting studies of brain function. Genetics 173: 2407-2410.
Kalendar R, Lee D, Schulman A H 2014. FastPCR software for PCR, in silico PCR, and oligonucleotide assembly and analysis. DNA Cloning and Assembly Methods, Methods in Molecular Biology, Svein Valla and Rahmi Lale (ed.), Humana Press, 1116: 271-302 (ISBN 978-1-62703-763-1).
DNA recommendations—further report of the DNA Commission of the ISFH regarding the use of short tandem repeat systems (1997) Forensic Sci Int 87: 179-184

The invention claimed is:

1. A method for genetically identifying and/or for distinguishing two or more animals of the species mouse, rat, hamster and zebrafish from different outbred strains, of the same outbred strain or of substrains of the same outbred strain, comprising the following substeps:
(a) using at least one DNA sample which is to be analyzed;
(b) selecting a set of loci for the DNA sample which comprises at least 5, preferably at least 6, 7 or 8, particularly preferably at least 9 or 10, most preferably at least 11 autosomal STR markers which are coamplified simultaneously in a reaction mix;
(c) selecting a set of loci for the DNA sample which comprises at least two Y-chromosomal STR markers which are coamplified simultaneously with the autosomal STR loci (b) in a reaction mix;
(d) using a set of oligonucleotide primer pairs in which each primer pair binds specifically to the flanking DNA segments of an STR marker from the sets of STR loci (b) and (c);
(e) coamplifying the loci selected for the set, the reaction product being a mixture consisting of amplified alleles of each of the coamplified STR loci of the sets (b) and (c);
(f) analyzing the allele mixture with the goal of determining and assigning the specific alleles for each of the amplified loci of the sets (b) and (c) for the given DNA sample;
the autosomal STR loci being characterized by a high value for the “polymorphism information content”, which is within the range of 0.30 to 0.99, preferably within the range of 0.50 to 0.99, particularly preferably within the range between 0.60 to 0.99.

2. The method as claimed in claim 1, characterized in that the DNA sample originates from at least one animal of the species mouse, rat, hamster or zebrafish or from a cell line of the species mouse, rat, hamster or zebrafish.

3. The method as claimed in claim 1, characterized in that at least two of the eleven autosomal STR loci (b) are selected from the group comprising the loci D1Mmu121 (SEQ ID NO: 51), D2Mmu008 (SEQ ID NO: 52), D3Mmu158 (SEQ ID NO: 53), D4Mmu155 (SEQ ID NO: 54), D5Mmu108 (SEQ ID NO: 55), D6Mmu120 (SEQ ID NO: 56), D7Mmu003 (SEQ ID NO: 57), D8Mmu127 (SEQ ID NO: 58), D9Mmu100 (SEQ ID NO: 59), D10Mmu043 (SEQ ID NO: 60), D11Mmu030 (SEQ ID NO: 61), D12Mmu056 (SEQ ID NO: 62), D13Mmu096 (SEQ ID NO: 63), D14Mmu074 (SEQ ID NO: 64), D15Mmu084 (SEQ ID NO: 65), D16Mmu030 (SEQ ID NO: 66), D17Mmu041 (SEQ ID NO: 67), D18Mmu069 (SEQ ID NO: 68) and D19Mmu008 (SEQ ID NO: 69).

4. The method as claimed in claims 1 and 3, characterized in that the set of eleven autosomal loci in step (b) comprises the loci D2Mmu008 (SEQ ID NO: 52), D3Mmu158 (SEQ ID NO: 53), D4Mmu155 (SEQ ID NO: 54), D6Mmu120 (SEQ ID NO: 56), D7Mmu003 (SEQ ID NO: 57), D8Mmu127 (SEQ ID NO: 58), D10Mmu043 (SEQ ID NO: 60), D13Mmu096 (SEQ ID NO: 63), D14Mmu074 (SEQ ID NO: 64), D16Mmu030 (SEQ ID NO: 66) and D18Mmu069 (SEQ ID NO: 68).

5. The method as claimed in claim 1, further comprising sex identification in the DNA sample of the individual in step (a) on the basis of the set of loci according to step (c).

6. The method as claimed in claim 5, further comprising distinguishing between Y chromosomes of the type Mus musculus musculus and of the type Mus musculus domesticus on the basis of the set of loci according to step (c).

7. The method as claimed in claim 5, characterized in that the locus for sex determination is a polymorphic STR locus of mouse that can distinguish Y-chromosomal haplotypes and is selected from a group of loci listed as follows: DYMmu001 (SEQ ID NO: 70), DYMmu002 (SEQ ID NO: 71) and DYMmu003 (SEQ ID NO: 72).

8. The method as claimed in claim 5, characterized in that the locus for sex determination is a polymorphic STR locus of rat that can distinguish Y-chromosomal haplotypes and is selected from a group of loci listed as follows: DYRno004 (SEQ ID NO: 73 and SEQ ID NO: 74), DYRno165 (SEQ ID NO: 75) and DYRno304 (SEQ ID NO: 76).

9. The method as claimed in claim 1, characterized in that a multiplex amplification with at least thirteen pairs of oligonucleotide primers, which comprises at least eleven autosomal and two Y-chromosomal STR loci, is carried out.

10. The method as claimed in claim 1, characterized in that the amplified alleles are separated from one another beforehand by means of an analytical or semipreparative method before the evaluation in step (f).

11. The method as claimed in claim 10, characterized in that at least one oligonucleotide primer of a primer pair is covalently coupled to a detection dye, preferably to a fluorescent dye.

12. The method as claimed in claim 11, characterized in that at least four different dye-coupled primers with four different detection dyes, preferably four different fluorescent dyes, are used.

13. The method as claimed in claim 1, characterized in that the assignment of the amplified alleles in step (f) is done on the basis of comparison with a size standard, the size standard being a mixture of DNA fragments of known size and/or a locus-specific mixture of known alleles.

14. A kit for the genetic identification and/or for the distinguishing of two or more animals from DNA extracts of individuals from wild-type populations, from different inbred or outbred strains, of the same inbred or outbred strain or of substrains of the same inbred or outbred strain of mouse, comprising:
(a) a set of oligonucleotide primer pairs in which each primer pair binds specifically to the flanking DNA segments of a locus from the set of STR loci and which comprises at least 5, preferably at least 6, 7 or 8, particularly preferably at least 9 or 10, most preferably at least 11 autosomal STR markers which can be coamplified simultaneously in a reaction mix;
(b) a set of oligonucleotide primer pairs which bind specifically to the flanking DNA segments of the Y chromosome and which comprises at least two, preferably three Y-chromosomal STR markers which can be coamplified simultaneously with the autosomal STR loci (a) in a reaction mix;
(c) reagents which are sufficient for carrying out at least one multiplex PCR; and
(d) a size standard which contains different DNA markers of a known fragment length.

15. The kit as claimed in claim 14, further comprising:
(e) a size standard which contains a locus-specific mixture of known alleles.

16. The kit as claimed in claim 14, characterized in that at least two of the eleven autosomal STR loci (a) are selected from the group comprising the loci D1Mmu121 (SEQ ID NO: 51), D2Mmu008 (SEQ ID NO: 52), D3Mmu158 (SEQ ID NO: 53), D4Mmu155 (SEQ ID NO: 54), D5Mmu108 (SEQ ID NO: 55), D6Mmu120 (SEQ ID NO: 56), D7Mmu003 (SEQ ID NO: 57), D8Mmu127 (SEQ ID NO: 58), D9Mmu100 (SEQ ID NO: 59), D10Mmu043 (SEQ ID NO: 60), D11Mmu030 (SEQ ID NO: 61), D12Mmu056 (SEQ ID NO: 62), D13Mmu096 (SEQ ID NO: 63), D14Mmu074 (SEQ ID NO: 64), D15Mmu084 (SEQ ID NO: 65), D16Mmu030 (SEQ ID NO: 66), D17Mmu041 (SEQ ID NO: 67), D18Mmu069 (SEQ ID NO: 68) and D19Mmu008 (SEQ ID NO: 69).

17. The kit as claimed in claim 14, characterized in that the set of eleven autosomal loci in step (a) comprises the loci D2Mmu008 (SEQ ID NO: 52), D3Mmu158 (SEQ ID NO: 53), D4Mmu155 (SEQ ID NO: 54), D6Mmu120 (SEQ ID NO: 56), D7Mmu003 (SEQ ID NO: 57), D8Mmu127 (SEQ ID NO: 58), D10Mmu043 (SEQ ID NO: 60), D13Mmu096 (SEQ ID NO: 63), D14Mmu074 (SEQ ID NO: 64), D16Mmu030 (SEQ ID NO: 66) and D18Mmu069 (SEQ ID NO: 68).

18. The kit as claimed in any of claim 14, characterized in that the set of Y-chromosomal loci (b) comprises the loci DYMmu001 (SEQ ID NO: 70), DYMmu002 (SEQ ID NO: 71) and DYMmu003 (SEQ ID NO: 72).
